(19) United States
(12) Patent Application Publication
(10) Pub. No.: US 2025/0331923 A1
(43) Pub. Date: Oct. 30, 2025

(54) System, Method and Apparatus for Creating a Portable Digital Twin of a Person's Anatomy

(71) Applicant: Vioma Health LLC, Ojai, CA (US)

(72) Inventor: Celine Marill Paganini, Ojai, CA (US)

(73) Assignee: Vioma Health LLC, Ojai, CA (US)

(21) Appl. No.: 19/189,320

(22) Filed: Apr. 25, 2025

Related U.S. Application Data
(60) Provisional application No. 63/638,432, filed on Apr. 25, 2024.

(51) Int. Cl. A61B 34/10; A61B 1/00; A61B 1/32

(52) U.S. Cl. CPC A61B 34/10; A61B 1/000096; A61B 1/32; A61B 2034/105

(57) ABSTRACT

A multifunctional diagnostic system includes a reusable speculum with a modular head and a portable digital twin station. The speculum includes a set of heated blades configured to comfortably open the vaginal canal, a numbing spray nozzle to anesthetize the cervix, and an integrated light source to illuminate the internal anatomy for clinical examination. The same speculum head supports a vaginal sweep using embedded imaging components and sensors to capture interior anatomical data. The captured data is wirelessly transmitted to a companion digital twin system that includes a processor, memory, and smart display. The system implements multi-spectral imaging configured to generate a digital twin of the patient's anatomy, which may be used for assessment, diagnosis, treatment planning, education, and analysis with artificial intelligence and extended reality platforms. A portable housing station serves as both a charging dock and sanitization unit.

### CROSS-REFERENCE TO RELATED APPLICATIONS

This application is a U.S. Non-Provisional Utility Patent Application entitled, “System, Method and Apparatus for Creating a Portable Digital Twin of a Person's Anatomy” which claims priority to co-pending U.S. Provisional Patent Application No. 63/638,432, filed on Apr. 25, 2024 entitled, “System, Method and Apparatus for Creating a Portable Digital Twin of a Person's Anatomy” the contents of which are hereby fully incorporated by reference.

### FIELD OF THE EMBODIMENTS

The field of the invention and its embodiments relate to systems and methods for a speculum and internal multi-spectral imaging, and more specifically to a system, method, and apparatus for creating a portable digital twin of a person's anatomy. In some embodiments, the system can be paired with artificial intelligence (AI) and extended reality (XR) technologies to assess, diagnose, and treat a person, or to support medical and health education through immersive and interactive learning experiences.

### BACKGROUND OF THE EMBODIMENTS

A speculum is a medical tool used upon humans and animals designed for examining the interior of body orifices inclusive of vaginal, rectal, and lower cervical regions. A speculum facilitates examinations by dilating an otherwise visually obstructed body region to provide a treating profession with a minimally obstructed view of the region being examined. While numerous examination instruments exist including, but not limited to, traditional speculum devices such as FemScop-like devices, a Callascope, and an Endoscope, our focus is on innovations to the reusable traditional speculum, which is sometimes referred to as a “duck-billed” speculum.

The traditional speculum has existed largely unchanged for approximately 150 years due to its inherent suitability for its task. Attempted alternatives are deemed by many to be functionally inferior, obstructive to examining physicians, unnecessarily intricate to operate, and/or prone to alternative-specific, patient experienced complications. Accordingly, the traditional speculum appears to be an enduring medical instrument likely to be a staple in the perceivable future.

Nonetheless, traditional speculum has numerous perceived shortcomings related principally to patient comfort that remain unresolved. For example, a reusable traditional speculum is formed from a conductive metal that imparts an unpleasant cold sensation upon insertion. Heating traditional metal speculums to body temperature prior to examination is not a widespread practice due to logistical and safety challenges. Specifically, heating and maintaining speculums at a body temperature in a medical setting can require specialized equipment and enhanced monitoring to ensure these instruments remain sterile and safe for use, especially considering bacterial growth concerns. Health care professionals typically do not use traditional lubricants on speculums during examinations, as lubricants may interfere with diagnostic accuracy. Instead, water is commonly used as a substitute; however, it does not function as an actual lubricant and often fails to reduce patient discomfort effectively. In contrast, the present speculum is designed to include an integrated numbing spray mechanism configured to deliver a topical anesthetic to the cervix, thereby helping to reduce pain and discomfort during procedures such as a Pap smear.

### Related Art

GB2391815A discloses a vaginal speculum for use with an embryo transfer catheter that includes a heat retaining material such as a gel. Specifically, an arm of the speculum includes a channel through which the gel flows, which warms the conductive portions of the speculum contacting the patient anatomy.

U.S. Pat. No. 11,805,993B2 discloses an assembly of a vaginal speculum and a reusable electric device. The reusable electric device comprises a housing with an aperture and including a camera module near a distal end of the housing and a wireless communication module near a proximal end of the housing.

U.S. Pat. No. 4,905,670A discloses a simple yet incredibly useful cervical videoscope has been provided which can easily be used by the doctor to examine the cervix for cancerous lesions or other abnormalities. Also, because of the small size of the camera there is sufficient space between the camera and the blades of the speculum for inserting forceps and other instruments that may need to be used.

CN 201847664U discloses a self-speculum for vagina, which belongs to the field of medical instruments including a disposable vaginal speculum, a colored camera, and a display device. This is a disposable vaginal speculum provided with an upper blade, an upper blade handle, a lower blade and a lower blade handle and is made of medical plastic material.

US20230337903A1 discloses a coaxial dilation speculum to access a human vagina with an enhanced light source to visualize the entire area. The disclosure contemplates using a camera instrument (FIG. 26) and provides for liquid ejection pathways to irrigate and clear surfaces via a jet of gas or spray of liquid.

Other notable prior art references include U.S. Pat. No. 4,971,036A, US20200107714, U.S. Pat. No. 10,127,665B1, and CN202313259U.

### SUMMARY OF THE EMBODIMENTS

What is needed is an improved speculum that retains the beneficial, time-proven elements of traditional designs while minimizing patient discomfort. Additionally, there is a need for a device that ensures accurate and objective documentation of findings, such as through high-resolution imaging, thereby reducing reliance on subjective diagnosis and minimizing unnecessary, duplicative procedures. The improved speculum may also incorporate sensors and light-based diagnostic tests to support real-time assessment of tissue characteristics, enhance clinical decision-making, and improve the overall quality and consistency of care.

Aspects of the present innovation enhance a reusable, traditional speculum for non-obtrusive heating of conductive traditional speculum contact surfaces to minimize patient discomfort while minimizing additional healthcare experienced device-use overhead and any practical concerns often associated with a heating of metallic medical instruments. Other aspects provide for a modular approach to detachably enhance functionality of the speculum to provide include integrated imaging components, ultrasound components, and a light source. Multi-spectral imaging and other components may be integrated into the medical instrument system to visually model an interior cavity and generate internal representations of a patient's anatomy. These visualizations may include 2D images, 3D models, and 4D dynamic renderings that capture both spatial structure and temporal changes, enhancing the ability to assess, document, and monitor anatomical conditions over time. The captured digital images are used to produce output products shareable with patients. In one embodiment, the output products may be referred to as a digital twin of the patient's anatomy. The output products visually highlight any discovered abnormalities or concerns. In embodiments, the innovative modular speculum can be utilized with or without modular components selectively permitting a treating physician to utilize enhanced heating or imagery capabilities or not depending on examination needs. In embodiments, the innovative speculum, along with its optional modular components available as part of the system, is a medical device designed for ease of cleaning and sterilization. Embodiments of the system may also provide storage and charging capabilities for components in junction with the sterilization.

One aspect of the discloser concerns a system for creating and presenting a digital twin of a patient's anatomy that includes a speculum and a digital twin system. The speculum includes a set of one or more blades, which are configured to be opened or closed. The speculum also includes at least one multi-spectral imaging component. The multi-spectral imaging component generates digital images of an interior of a patient's anatomy when the speculum is used on a patient and when the one or more blades are opened. The speculum also includes a transceiver configured to wirelessly transmit the digital images over a network. The speculum is a portable device that operates without physical cables connecting the imaging component to an external computing device. The external computing device may be integrated into a multifunctional housing unit that also serves as a cleaning device and charging station for the speculum, enabling streamlined storage, disinfection, and power management between uses. The digital twin system is configured to receive digital images from the imaging component and to process those images, along with other data points obtained from integrated sensors-such as pressure, temperature, acidity, and results from light-based tests-to create a comprehensive digital twin of the patient. This multi-modal data integration enables a more accurate and dynamic representation of the patient's anatomical and physiological state. The digital twin is a visual, anatomical representation of at least a portion of the patient's anatomy generated through the use of the speculum head or other optional attachment heads. The system is not limited to internal examinations and may also be used for external anatomical assessments, allowing for versatile clinical applications across various regions of the body. The digital twin system also outputs the digital twin to a user of the digital twin system.

Another aspect of the disclosure is a system for creating and presenting a digital twin of a patient's anatomy, which includes a speculum, a case, and a digital twin system. The speculum includes at least one imaging component, wherein the imaging component is configured to generate digital images of a patient's internal anatomy. In some embodiments, imaging of internal structures may also be achieved using attachment heads that are designed for external use, providing flexibility in how anatomical data is captured based on clinical needs. The speculum includes an upper leaf, an upper petiole, a lower leaf, a lower petiole, a hinge, and a viewing window. The imaging component is positioned to not appreciably impede a physician's view via the viewing window when the speculum is used.

The speculum also includes a transceiver configured to wirelessly transmit the digital images over a low-power network. In one embodiment, the speculum is a portable device that lacks physical cables connecting the imaging component to an external computing device. Thus, the speculum is cordless. The case includes a compartment configured to secure the speculum, when not in use. The speculum includes a rechargeable, portable power source, which is automatically charged from a power source associated with the case when the speculum is contained in the compartment.

In some embodiments, the case includes a multifunctional display, a low-powered transceiver, a high-distance transceiver, and a full keyboard. In addition to serving as a visual interface, the display functions as a control panel for operating the system, enabling user interaction and control of imaging and data processing functions directly from the case. The low-power transceiver communicates over the low-power network to receive the digital images. The low-power network can be a personal area network and the low-power transceiver can be a BLUETOOTH one. The high distance transceiver can communicate with the internet, which enables it to communicate with the remotely located digital twin system.

The digital twin system includes a processor, memory, and transceiver. The digital twin system can receive the digital images captured by the imaging component conveyed over the internet from the case to the digital twin system and can process the digital images to create a digital twin of the patient. The digital twin is a visual, anatomical representation of at least a portion of the patient's anatomy, generated through the use of the speculum head or other optional head add-ons. These interchangeable components allow for flexible examination of both internal and external anatomical regions based on the clinical application. The digital twin is conveyed over the internet to the case. The visually displays the digital twin on the case display. In one embodiment, the case can run a client application in communication with a digital twin application running on the digital twin system, which operates in real time or near real time. Thus, digital images captured by the speculum head or other interchangeable heads are transmitted to the case over the low-powered network, sent to the digital twin server via the internet for processing, and returned in their processed form to be displayed on the case's integrated display screen.

Another aspect of the disclosure teaches a system for creating and presenting a digital twin of a patient's anatomy comprising a speculum and a case. The speculum includes a set of one or more blades, which are configured to be opened or closed and at least one imaging component. The imaging component generates digital images of an interior of a patient's anatomy when the speculum is used on a patient and when the one or more blades are opened. The speculum includes a first low-power transceiver configured to wirelessly transmit the digital images over a personal area network (PAN), such as a BLUETOOTH one. The speculum is a portable device that lacks physical cables connecting the imaging component to an external computing device. The case includes a compartment configured to secure the speculum when not in use, a case display, and a low-power transceiver configured to communicate over the personal area network to receive the digital images.

In some aspects, the techniques described herein relate to a system for creating and presenting a digital twin of a patient's anatomy including: a speculum including: a set of one or more blades, which are configured to be opened or closed; at least one imaging component, wherein said imaging component is configured to generate digital images of an interior of a patient's anatomy, when the speculum is used on a patient and when the one or more blades are opened; and a transceiver configured to wirelessly transmit the digital images over a network, wherein the speculum is a portable device that lacks physical cables connecting the imaging component to an external computing device; and a digital twin system including a processor, memory, and transceiver, wherein said digital twin system is configured to: receive the digital images of the imaging component; to process the digital images to create a digital twin of the patient, wherein the digital twin is a visual, anatomical representation of at least a portion of the patient's anatomy examined through use of the speculum; and output the digital twin to a user of the digital twin system.

In some aspects, the techniques described herein relate to a system, wherein the speculum is a two-bladed vaginal speculum, which further includes: an upper leaf; an upper petiole; a lower leaf; a lower petiole; a hinge; and a viewing window, wherein the imaging component is positioned to not appreciably impede a physician's view via the viewing window when the speculum is used.

In some aspects, the techniques described herein relate to a system, wherein the imaging component is modular and configured to be detachably coupled and interchangeably utilized with at least one speculum head or an ultrasound head, the speculum head including various sizes of the one or more blades and different diameters associated with an opened state of the blades.

In some aspects, the techniques described herein relate to a system, wherein the at least one imaging component is an ultrasound component configured to produce ultrasound-based images.

In some aspects, the techniques described herein relate to a system, wherein the speculum further includes: a heater, which warms the set of one or more blades to within five percent of a body temperature of a patient; a fail-safe component including a temperature-activated limit switch configured to interrupt power to the heater when a sensed temperature exceeds a predefined safety threshold, thereby preventing the heater from overheating the set of one or more blades; and a light source configured to emit both visible light in a wavelength range of approximately 400 to 700 nanometers and near-infrared (NIR) light in a wavelength range of approximately 700 to 1000 nanometers.

In some aspects, the techniques described herein relate to a system, further including: a case including a compartment configured to secure the speculum, when not in use, wherein the speculum includes a portable power source, which is automatically charged from a power source associated with the case, when the speculum is contained in the compartment.

In some aspects, the techniques described herein relate to a system, wherein the case is a portable case used to carry and secure the speculum.

In some aspects, the techniques described herein relate to a system, wherein the case includes a first and second case transceiver, wherein the transceiver of the speculum is a low-power one able to wirelessly transmit the digital images over a personal area network having a low-power range of less than 100 yards, wherein the first case transceiver receives the digital images over a connection of the personal area network, wherein the second case transceiver has a range greater than that of a low power range, which conveys the digital images to the transceiver of the digital twin system, which is further away from the speculum than the low-power range.

In some aspects, the techniques described herein relate to a system, further including: a sanitizer, which sanitizes the speculum when placed in the compartment using one or more sanitization modalities, including but not limited to ultraviolet radiation, heat, ozone, or chemical disinfectants; and a numbing spray including a topical anesthetic to contact a portion of the patient's anatomy to reduce discomfort during examination or sample collection.

In some aspects, the techniques described herein relate to a system, wherein the case includes a user interface including: a display screen, one or more input controls, and a keyboard, the user interface being configured to display the digital images and receive user input for operating the system.

In some aspects, the techniques described herein relate to a system, wherein the digital twin is a three-dimensional image.

In some aspects, the techniques described herein relate to a system, wherein the digital twin is an image derived from a plurality of different ones of the digital images.

In some aspects, the techniques described herein relate to a system, wherein the digital twin visually identifies at least one potential anomaly, which is automatically determined from content of the digital images.

In some aspects, the techniques described herein relate to a system, wherein the digital twin system performs real time image processing of the digital images that are received in real time.

In some aspects, the techniques described herein relate to a system for creating and presenting a digital twin of a patient's anatomy including: a speculum including: a housing including: a handle; a control interface; an opening; a release mechanism configured to eject a container retaining a topical anesthetic from a compartment of the housing, the topical anesthetic configured to be dispensed from the housing when the container is retained in the housing; at least one imaging component, wherein said imaging component is configured to generate digital images of an interior of a patient's anatomy, when the speculum is used on a patient; a set of adjustable blades, the adjustable blades are configured to be oriented in an open position and a closed position; a support structure connected to the set of blades, the support structure including: a protrusion structure configured to be retained in the opening of the housing; and a rotation mechanism configured to rotate the set of adjustable blades; a pivot structure configured to orient the support structure along a y-axis; and a viewing window, wherein the imaging component is positioned to not appreciably impede a physician's view via the viewing window when the speculum is used; and a transceiver configured to wirelessly transmit the digital images over a low-power network, wherein the speculum is a portable device that lacks physical cables connecting the imaging component to an external computing device; a case including: a compartment configured to secure the speculum, when not in use, wherein the speculum includes a portable power source, which is automatically charged from a power source associated with the case, when the speculum is contained in the compartment; a case display; a low-power transceiver configured to communicate over the low-power network to receive the digital images; a high distance transceiver configured to communicate with an internet; a digital twin system including a processor, memory, and transceiver, wherein said digital twin system is configured to: receive the digital images captured by the imaging component conveyed over the internet from the case to the digital twin system; to process the digital images to create a digital twin of the patient, wherein the digital twin is a visual, anatomical representation of at least a portion of the patient's anatomy examined through use of the speculum; and convey the digital twin over the internet to the case, wherein the case is further configured to represent the digital twin on the case display.

In some aspects, the techniques described herein relate to a system for creating and presenting a digital twin of a patient's anatomy including: a speculum including: a set of blades, which are configured to be opened or closed; at least one imaging component, wherein said imaging component is configured to generate digital images of an interior of a patient's anatomy, when the speculum is used on a patient and when the set of blades are opened; and a first low-power transceiver configured to wirelessly transmit the digital images over a personal area network, wherein the speculum is a portable device that lacks physical cables connecting the imaging component to an external computing device; a case including: a compartment configured to secure the speculum, when not in use, wherein the compartment is further configured to charge the speculum and sanitize the speculum using one or more sanitization modalities including one or more of ultraviolet radiation, heat, ozone, or a chemical disinfectant; a case display; and a low-power transceiver configured to communicate over the personal area network to receive the digital images.

In some aspects, the techniques described herein relate to a system, wherein the personal area network is a Bluetooth network, wherein the case further includes: an internet transceiver configured to connect the case to the internet.

In some aspects, the techniques described herein relate to a system, wherein the speculum is a two-bladed vaginal speculum, which further includes: one or more heating elements traversing at least a portion of a length of the set of blades; a temperature-activated limit switch configured to interrupt power to the one or more heating elements when a temperature measurement from a temperature sensor exceeds a predefined safety threshold, thereby preventing the heating element from overheating the set of blades; and a viewing window, wherein the imaging component is positioned to not appreciably impede a physician's view via the viewing window when the speculum is used.

In some aspects, the techniques described herein relate to a system, wherein the speculum includes a portable power source, which is automatically charged from a power source associated with the case when the speculum is contained in the compartment, wherein the case is a portable case used to carry and secure the speculum.

In some aspects, the techniques described herein relate to a system, further including: a digital twin system including a processor, memory, and transceiver, wherein said digital twin system is configured to: receive the digital images of the imaging component; to process the digital images to create a digital twin of the patient, wherein the digital twin is a visual, anatomical representation of at least a portion of the patient's anatomy examined through use of the speculum; and output the digital twin to a user of the digital twin system.

### BRIEF DESCRIPTION OF THE DRAWINGS

FIG. 1A depicts a system for generating and utilizing a digital twin, in accordance with embodiments of the present invention.

FIG. 1B depicts a side plan view of an example speculum implementing the system of FIG. 1A, in accordance with embodiments of the present invention.

FIG. 1C depicts a side plan view of the example speculum of FIG. 1A with a power source, in accordance with embodiments of the present invention.

FIG. 1D depicts a front plan view of the example speculum of FIG. 1A with a viewing window, in accordance with embodiments of the present invention.

FIG. 2 depicts a display of a device showing an operational sequence that may be performed when running a digital twin application with the system of FIG. 1A, in accordance with embodiments of the present invention.

FIG. 3 is a block diagram of a computing device implemented by the system of FIG. 1A, in accordance with embodiments of the present invention.

FIG. 4 is a front plan view of another example speculum implementing the system of FIG. 1A with an unassembled housing of the smart speculum system including a housing with a handle and a control interface, in accordance with embodiments of the present invention.

FIG. 5 is a front top perspective view of the unassembled housing of the smart speculum system of FIG. 4 including the housing with the handle, the control interface, and an opening configured to receive a support structure of a set of blades, in accordance with embodiments of the present invention.

FIG. 6 is a left side plan view of the unassembled housing of the smart speculum system of FIG. 4 including the housing with the handle, the control interface, and a release mechanism with a switch to be depressed, in accordance with embodiments of the present invention.

FIG. 7 is a left side exploded view of the unassembled housing of the smart speculum system of FIG. 4 including the housing with the handle, the control interface, and a release mechanism with a switch to be depressed and to eject a cap and a container retaining a topical anesthetic from a compartment of the housing, the topical anesthetic configured to be dispensed from the housing when the container is retained in the housing, in accordance with embodiments of the present invention.

FIG. 8 is a front perspective view of a support structure connected to a set of blades oriented in a closed position, the support structure is configured to be removably connected to the housing of FIGS. 4-7, in accordance with embodiments of the present invention.

FIG. 9 is a front perspective view of the support structure of FIG. 8 connected to a set of blades oriented in an open position, the support structure has a protrusion structure configured to be retained in the opening of the housing of FIGS. 4-7, in accordance with embodiments of the present invention.

FIG. 10 is a front perspective view of the support structure of FIG. 9 connected to a set of blades oriented in an open position, the support structure has a rotation mechanism configured to rotate the set of adjustable blade in a first direction and in a second opposite direction, in accordance with embodiments of the present invention.

FIG. 11 is a rear perspective view of the support structure of FIG. 9 connected to a set of blades oriented in a closed position, the support structure has a pivot structure configured to orient the support structure along the y-axis and has one or more heating elements traversing at least a portion of the length of the set of blades, in accordance with embodiments of the present invention.

FIG. 12 is a rear perspective view of the support structure of FIG. 9 connected to a set of blades oriented in an open position, in accordance with embodiments of the present invention.

FIG. 13 is a front perspective view of the assembled housing of FIGS. 4-7 connected to the support structure of FIGS. 8-12 with the set of blades oriented in an open position, in accordance with embodiments of the present invention.

FIG. 14 is a left side plan view of the housing of FIGS. 4-7 connected to the support structure of FIGS. 8-12 forming an assembled speculum with the set of blades oriented in an open position, in accordance with embodiments of the present invention.

FIG. 15 is a left side plan view of the assembled speculum of FIG. 14 with a pivot structure configured to orient the set of blades and the support structure along the y-axis from about 0 degrees to about 90 degrees, in accordance with embodiments of the present invention.

FIG. 16 is a front perspective view of a portable carrying case in a closed position with a telescoping handle and configured to retain the system of FIG. 1A, in accordance with embodiments of the present invention.

FIG. 17 is a rear perspective view of a portable carrying case of FIG. 16 in a closed position and having foldable legs retracted, in accordance with embodiments of the present invention.

FIG. 18 is a perspective view of a portable carrying case of FIG. 16 in an opened position with a display and a retractable control panel retained in the portable carrying case and having foldable legs extended, in accordance with embodiments of the present invention.

FIG. 19 is a perspective view of a portable carrying case of FIG. 16 in an opened position with a display and a retractable control panel of FIG. 18 with a charging dock and a power source, in accordance with embodiments of the present invention.

FIG. 20 is a perspective view of a plurality of removable probe attachments configured for use with the speculum of FIG. 4, in accordance with embodiments of the present invention.

FIG. 21 is a perspective view of a plurality of removable reusable speculum heads of differing sizes and configured for use with the speculum of FIG. 4, in accordance with embodiments of the present invention.

### DESCRIPTION OF THE PREFERRED EMBODIMENTS

The preferred embodiments of the present invention will now be described with reference to the drawings. Identical elements in the various figures are identified with the same reference numerals.

Reference will now be made in detail to each embodiment of the present invention. Such embodiments are provided by way of explanation of the present invention, which is not intended to be limited thereto. In fact, those of ordinary skill in the art may appreciate upon reading the present specification and viewing the present drawings that various modifications and variations can be made thereto.

As used herein, the singular forms “a,” “an,” and “the,” are intended to include the plural forms as well, unless the context clearly indicates otherwise.

As used herein in the specification and in the claims, the phrase “at least one,” in reference to a list of one or more elements, should be understood to mean at least one element selected from any one or more of the elements in the list of elements, but not necessarily including at least one of each and every element specifically listed within the list of elements and not excluding any combinations of elements in the list of elements. This definition also allows that elements may optionally be present other than the elements specifically identified within the list of elements to which the phrase “at least one” refers, whether related or unrelated to those elements specifically identified. Thus, as a non-limiting example, “at least one of A and B” (or, equivalently, “at least one of A or B,” or, equivalently “at least one of A and/or B”) can refer, in one embodiment, to at least one, optionally including more than one, A, with no B present (and optionally including elements other than B); in another embodiment, to at least one, optionally including more than one, B, with no A present (and optionally including elements other than A); in yet another embodiment, to at least one, optionally including more than one, A, and at least one, optionally including more than one, B (and optionally including other elements); etc.

As used herein, an “embodiment” means that a particular feature, structure, or characteristic is included in at least one or more manifestations, examples, or implementations of this invention. Furthermore, the particular features, structures or characteristics may be combined in any suitable manner, as would be apparent to a person having ordinary skill in the art. Combinations of features of different embodiments are meant to be within the scope of the invention, without the need for explicitly describing every possible permutation by example. Thus, any of the claimed embodiments can be used in any combination.

The phrase “and/or,” as used herein in the specification and in the claims, should be understood to mean “either or both” of the elements so conjoined, e.g., elements that are conjunctively present in some cases and disjunctively present in other cases. Thus, as a non-limiting example, a reference to “A and/or B,” when used in conjunction with open-ended language such as “comprising” can refer, in one embodiment, to A only (optionally including elements other than B); in another embodiment, to B only (optionally including elements other than A); in yet another embodiment, to both A and B (optionally including other elements), etc. As used herein, the word “include,” and its variants, is intended to be non-limiting, such that recitation of items in a list is not to the exclusion of other like items that may also be useful in the compositions and methods of this technology. Similarly, the terms “can” and “may” and their variants are intended to be non-limiting, such that recitation that an embodiment can or may comprise certain elements or features does not exclude other embodiments of the present technology that do not contain those elements or features.

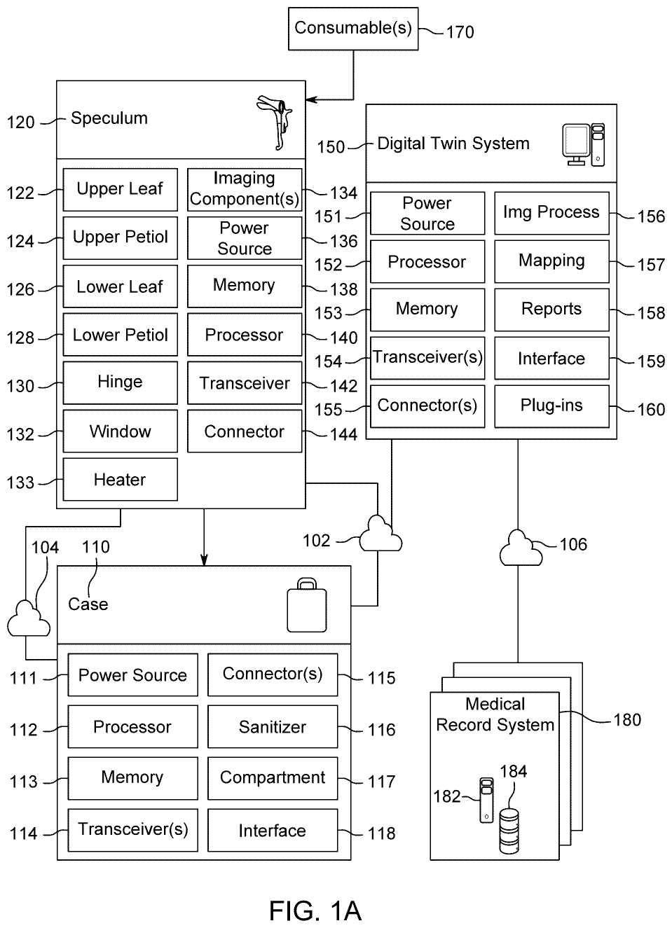

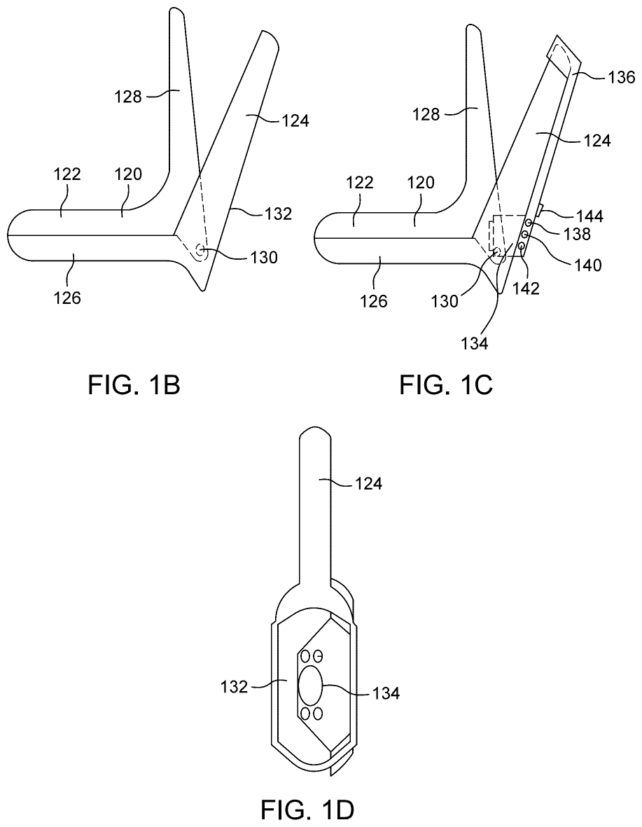

FIG. 1A depicts a system for generating and utilizing a digital twin, in accordance with aspects of the disclosure. As shown, speculum 120 utilizes a set of imaging components 134 to produce digital images of a being's anatomy, which are conveyed and processed by the digital twin system 150. Imaging components 134 can be integrated and/or modularly equipped to the speculum 120 in embodiments. An imaging component 134 may include a light, camera, an ultrasound component, and the like. In embodiments, multiple different imaging techniques can be utilized separately or combined to create the digital twin. Different views of the digital twin can be generated, which include two-and three-dimensional image outputs. Imaging outputs are not limited to the cervix and may also include visualizations of the vaginal walls and other internal anatomy. These outputs may comprise, for example, three-dimensional saline-contrast sonovaginocervicography (SVC) views, transverse and coronal plane views of the cervix and surrounding tissues, virtual speculoscopy views, combinations thereof, as well as non-conventional or enhanced renderings for broader diagnostic assessment. For example, image processing 156 can incorporate data imagery and data from a magnetic resonance imaging (MRI) machine, during the construction of the digital twin and/or one or more of its integrated views. Similarly, x-ray imagery can be mapped (157) to images output from component(s) 134 during a creation of the digital twin. In short, a true “digital twin” having multiple, selectable optional views are generated in embodiments. Of course, in embodiments, digital twin images are composed from raw data obtained from imaging component 134 without additional imagery, such as that from an MRI or X-ray machine.

In one embodiment, the system comprises a medical device including one or more imaging components configured to capture anatomical data of a patient. For example, the imaging components may include a high-resolution RGB camera, a miniaturized ultrasound probe, or a combination thereof, positioned within or on a reusable speculum head. The captured data may include image sequences, sonographic slices, 3D volumetric data, or multimodal inputs combining visual and acoustic signatures. The imaging components may be modular or fixed and are operatively coupled to a processor.

The processor, which may be located within a portable medical case or an attached computing module, receives the anatomical data in real time. The processor executes software instructions that extract anatomical features and generate a digital twin model of at least a portion of the patient's anatomy. The digital twin may be rendered as a 3D mesh model, a voxel-based reconstruction, or a time-series anatomical simulation. In some embodiments, the digital twin incorporates anatomical landmarks (e.g., cervix, vaginal canal, cervical os) automatically segmented from imaging inputs.

The digital twin model is then processed by an artificial intelligence engine, which comprises one or more neural networks. The AI engine may be implemented using a convolutional neural network (CNN) for image-based segmentation or lesion detection, a transformer model for longitudinal or multimodal signal analysis, a recurrent neural network (RNN) for interpreting dynamic anatomical changes over time, or a graph neural network (GNN) to model spatial relationships between anatomical substructures. In one implementation, a CNN may analyze the pixel-wise intensity values of the cervix to identify potential abnormalities, such as ectropion, neoplastic changes, or polyps. In another example, a transformer may process a time-series of sonographic frames to infer cyclical physiological patterns or inflammatory progression.

Importantly, the AI engine is configured to improve diagnostic accuracy over time. In certain embodiments, the system supports federated learning, wherein model parameters are updated based on distributed training data from multiple devices or clinical sites, without transmitting raw patient data to a centralized location. Clinical outcome data, such as biopsy confirmations or follow-up diagnostics, may be used to refine the AI engine's inference capabilities. Alternatively or additionally, reinforcement learning may be employed, where feedback loops based on clinician actions or patient outcomes inform future model predictions. These learning processes may occur asynchronously in the cloud, with periodic parameter synchronization back to the local device.

The inference outputs, including diagnostic insights or suggested follow-up steps, are displayed to a user via a graphical interface. The interface may be located on the portable medical case (e.g., smart touchscreen), on a paired tablet, or streamed to a cloud-based physician dashboard. The diagnostic insights may include annotated images, probability heatmaps, confidence scores, or flagged regions of interest. In an exemplary use case, a clinician performing a cervical exam may view a real-time, AI-enhanced overlay on the digital twin that highlights areas requiring biopsy or further imaging.

The system supports hybrid processing architecture, wherein low-latency Al inference is performed locally on the processor housed within the portable case using an embedded GPU or AI accelerator. This supports real-time operation in low-connectivity environments (e.g., rural clinics, field settings). Higher-order analytics, such as cross-patient trend modeling or AI model retraining, are deferred to a cloud-based processing environment, which may include GPU clusters or model orchestration servers.

The digital twin model is continuously updated during examination in response to new image frames or sensor readings. This enables a dynamic visualization of anatomical changes (e.g., tissue deformation, motion compensation, lesion evolution). In some embodiments, the digital twin may integrate extended reality (XR) features, including AR overlays of diagnostic insights or VR simulation of patient-specific anatomy for educational purposes.

FIG. 1B illustrates speculum 120 is configured to be communicatively linked to case 110, digital twin system 150, and/or a portable computing device via network 102 and 104. The digital twin system 150 may be connected via network 106 to a set of medical record system(s) 180, each of which can have a dedicated server 182 and repository 184. Systems 180 may include a hospital's system, an electronic medical records (EMR) and/or an Electronic Health Record system (EHR), where communications implement appropriate security measures, laws, restrictions, and standards. Plug-ins 160 tailored to specific ones of these systems 180 can help tailor output accordingly. For example, plug-ins 160 can exist for various popular EHR software platforms including, but not limited to, those provided by EPIC SYSTEMS CORPORATION, ORACLE CERNER, MEDITECH, EVIDENT, ALTERA DIGITAL HEALTH, MEDHOST, and the like.

FIG. 1C illustrates speculum 120 may be structured in that it includes an upper leaf 122, upper petioles 124, lower leaf 126, hinge 130, and viewing window 132, embodiments of which are reflected in FIGS. 1B-1D. In one embodiment, an integrated heater 133 is provided for patient comfort. In embodiments, speculum 120 may be considered a “smart device”, which includes a power source 136, memory 138, processor 140, transceiver 142, and one or more connectors 144, in addition to inclusion of the set of imaging components 134, one or more sensors, and/or numbing spray for the pap smear. In a preferred embodiment, the speculum 120 does not have a physical cable connecting it directly to a separate computing device, as such a physical cable can be highly cumbersome in use. Further, integrated cables diminish the portability of such a device. Other embodiments, however, may utilize a dedicated cable for communication with a remote system, such as system 150, which still allows for enhanced imaging referred to herein, as the digital twin.

In a portable embodiment, the speculum 120 can be paired to fit within a portable case 110. When not in use, the speculum 120 fits snuggly into a protective component 117. The case 110 can include a sanitizer 116. Speculum may communicate over a localized network 104 connection, such as a BLUETOOTH connection, with the case 110, which may have additional networking capabilities, enabled by a WIFI or cellular transceiver 114. Such a configuration has the advantage of utilizing low-power consumption for localized communications, which minimizes a drain of power source 136 and extends time of use between charges. In another embodiment, a removable memory 138 may capture images, which is transferred to other systems, such as memory 113 and/or memory 113 when convenient. In embodiments, real-time processing and imaging can occur, where a case-equipped interface 118, inclusive of visual display can be used to view raw images processed by speculum 120. Other remote monitors can be utilized, such as those connecting to a case's 110 connector 115 and/or one that is otherwise connected wirelessly to network 104 or 102. In a further embodiment, the case 110 is connected over a network 102 to a digital twin system 150 with image processing 156 capabilities, such that processed images are displayable in real-time or near-real time via interface 118 or an otherwise connected display. In one embodiment, the case 110 and/or speculum 120 can be designed to directly interface with a portable computer, such as a notebook computer, a tablet, and/or a smart phone, which provides additional input/output mechanisms in lieu and/or in addition to those provided via interface 118.

Referring to FIGS. 1B-1D, speculum 120 may be implemented as a vaginal speculum that includes a color camera or ultrasonic imaging component 134, a display device (such as interface 118 or a display of a connected portable computing device/display). Vaginal speculum 120 is designed for use in a variety of settings, including but not limited to hospitals, doctors' offices, home environments, off-site client visits, and educational or training settings. The device includes structural components such as an integrated upper leaf 122, upper petiole 124, lower leaf 126, and lower petiole 128. The upper leaf 122 and lower leaf 126 are joined by a pivoting, hinged, or spring connection 130. In some embodiments, alternative opening mechanisms, such as a spiral-expansion design, may be employed to widen or close the leaves and create an adjustable viewing window, enhancing visualization and patient comfort during examinations or procedures. Upper petiole 124 is provided with a viewing window 132 (as shown in FIG. 1D). Integration of the lower leaf 126, lower petiole 128 can be used in a hand-held state. The imaging component 134 (FIGS. 1C and 1D) can include a camera and/or ultrasonic component, a housing, a lens eleven, an output pathway, and a lighting lamp. The housing is placed in the body, such that a front end of the lens and lighting lamp is placed for view capture via the viewing window 132, while minimally disrupting a view via window 132 needed by a clinician, nurse, or physician. In various embodiments, imaging component 134 is detachably coupled, and speculum 120 can be utilized conventionally (without image capability), when detached. Further, different image components 134 can be detachably coupled, such as one including a camera and another including an ultrasonic imager. In embodiments, different modules can be attached/detached while the speculum 120 is in use (i.e., dilating a patient). Different visual imagers, having different focal lengths, fidelities, spectrum ranges, and the like can be utilized. In one embodiment, a single imaging component 134 can include both a camera and an ultrasound imager.

The heater 133 warms the blades or arms (leaf 122, leaf 126) that contact a patient's anatomy to be at or near body temperature. In one embodiment, the blades (122, 126) can be conductive elements having integrated heating elements. Further, one or more temperature sensors can be integrated to determine a temperature of the blades 122, 126 and/or a temperature of the human body contact point(s) to ensure warming to at or near a body temperature is achieved. Power for the heating element 133 can be provided from power source 136. In embodiments, application of lubricants (one variant of consumable 170) can ensure smooth heating/cooling.

Consumables 170 include sanitary covering(s), lubricants, cloths, and the like. In one embodiment, a set of heads for the blades (leaf 122, 126) can fit over the outer surface for sanitary and comfort purposes. Further, the heads can alter the aperture size of the speculum 120.

In a preferred embodiment, the speculum 120 is a duck-bill shaped device made of medical grade materials able to be sanitized and reused. In embodiments, the speculum 120 can include a removable/disposable head and a permanent base. These heads can be intentionally different shapes and sizes, for the comfort of the patient. Speculum can have two blades, as shown in FIGS. 1B-1D, or may be implemented with one or three blades in other embodiments.

A two-bladed, or bivalve speculum 120, also referred to as CUSCO's speculum) is type often used by gynecologists to examine the vagina and cervix. Upon insertion, the blades open, to expose the inside of the vagina and cervix for viewing via window 132. Imaging component(s) are arranged to properly capture images of the inside of the patient's anatomy, while retaining unobstructed viewing via window 132.

In one embodiment, speculum 120 can be a HUFFMAN speculum, which is a long, thin speculum that is narrower than a regular speculum, often utilized for examination of teenaged girls not yet sexually active. In one embodiment, speculum 120 can be a PEDERSON speculum, with blades narrower than those used for a regular adult, but larger than those of a HUFFMAN speculum. A PEDERSON speculum is often used for teenage girls that are sexually active. In still another embodiment, speculum 120 can be a GRAVES speculum, which has the widest blades of any speculum. Gynecologists often use a GRAVES speculum to examine adult women. In one embodiment, the speculum 120 can be an anal speculum, sometimes called an anoscope, which is a tube-shaped instrument that widens the opening of the anus.

Speculum 120 is not limited to use upon human patients but may in embodiments be configured as a veterinary speculum for animal care. Veterinary speculums 120 vary significantly in size, as they are adapted for the bodies of the various sizes of animals for which they are used.

With reference to FIG. 1A, the digital twin system 150 may be a computing device that includes a power source 151, a processor 152, a memory 153, a transceiver 154, and a set of connectors 155. Further, the digital twin system 150 can include the image processor 156, a mapping component 157, a report component 158, a user interface 159 and related components, as well as a set of plug-ins 160.

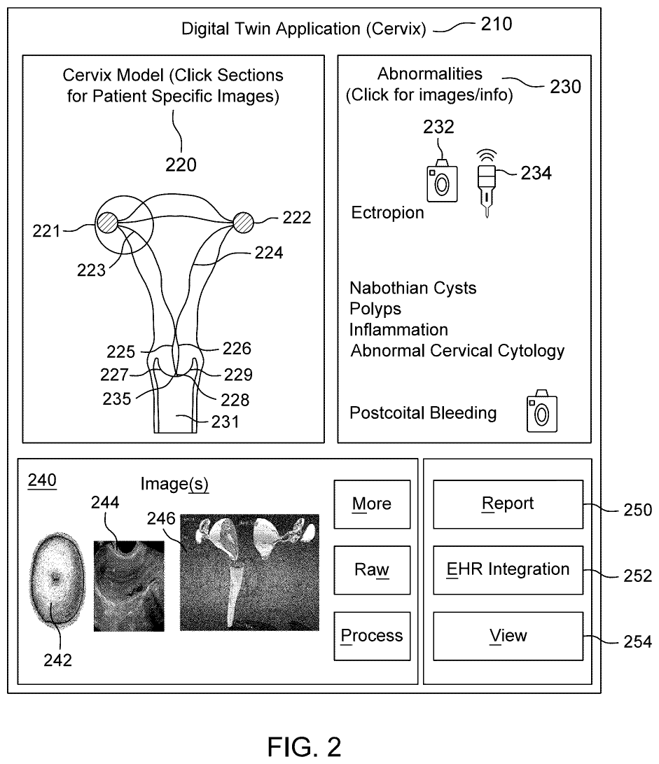

With reference to FIG. 2 the digital twin system 150 can include a digital twin application 210. The application 210 can run locally upon a computing device or can run at least partially within a server and be provided over a network 102, 106, to a browser or other client-side interface. As noted, this interface integrated into case 110 or a computing device proximate to speculum 120, when used remote from a physical location of system 150. The application 210 can include numerous appearances and modules, some of which are detailed in simplistic fashion in FIG. 2.

Various use modules, such as for use in examining a cervix can be integrated into application 110. When used to examine an anus, different modules and screens can be active. Further, modules can be adaptive and representative for diverse types of patients, such as a human, child, or animal (in veterinary settings). A model of a region associated with a digital twin can be presented in model section 220. A cervix model is shown, which provides for model components consistent with an anatomy of the patient, which includes adnexa 221, ovary 222, fallopian tube 223, uterus 224, columnar cell area 225, cervical canal 226, squamous cell area 227, squamo-columnar junction 228, cervical OS 235, cervix 229, and vaginal canal 231. A selection of each component can automatically bring up details application thereto. The various model components can bring up patient specific information on each, as well as additional information useful for the application's user 210.

In various embodiments, users of application 210 can include physicians, veterinarians, health care professionals, and patients themselves. Accordingly, diverse types of users may be exposed to different information. Various security measures can apply to different data elements and the respective users of the applications, to ensure that patient privacy, physician draft notes, hospital regulations, and patient record laws and procedures are properly respected.

In one embodiment, the image processing 156 functions performed by system 150 can automatically highlight and/or determine a set of potential abnormalities.

In a further embodiment, artificial intelligence features, such as the use of a Hidden Markov Model (HMM) trained with relevant data sets, may be utilized not only for abnormality identification and detection but also to assist in generating personalized treatment plans based on the analyzed findings. Each abnormality reported may have a set of associated images, which are those taken or derived from imaging component 134. For example, an abnormality related to ectropion can have one camera image 242 and one ultrasound image 234 associated with it. Clicking on either icon 232, 234 can bring up a related set of images highlighting the problem, as viewed in the digital twin. Some abnormalities may have no images associated, as they can be determined from visual inspection and/or from notes added by health care professionals. In various embodiments, images obtained from other sources, such as an x-ray or MRI, can be integrated next to an abnormality. The images mentioned, in one embodiment, may be digitally processed images generated by the image processor, which may more clearly provide visual imagery when contrasted to raw images, which are often challenging to read/interpret.

It should be noted that in one embodiment, various abnormalities and information contained therein can be mapped to various model components 221-229, 231, and 235 by mapping module 157. Model components can also be mapped by module 157 to digital twin creations. Such a mapping simplifies information indexing within database implementations, which are contemplated herein.

Image section 240 of application 210 can interactively react to selections in other sections 220 and 230. For example, a camera based raw image 242 from imaging component can be presented when that component has focus in section 220. One or more additional images, such as ultrasound image 244 can also be displayed. Further, a three-dimensional model 246, which is also referred to as the digital twin, is displayed. This model 246 can be an interactive one, which was formed or derived from a set of images and other available information. The digital twin 246 thus varies significantly from patient-to-patient, as each is uniquely generated from a set of images taken for that specific patient. Image 246 may be automatically generated by image processing component 156. In embodiments images in section 244 can be filtered to restricted to raw images, generated images, and the like. Further, in embodiments, a specific selected image or image set can be expressly processed, which may highlight related potential abnormalities shown in section 230.

Application 210 can generate one or more reports 250 used by healthcare professionals and patients alike. In one embodiment, EMR and EHR integration can be selected 252, which may rely upon platform specific plugins 160, A Pls, and the like. The digital twin application 210 can also have multiple different interface specific views 254. For example, although a model view for section 220 is shown, a 3D model view (image 246) could be presented in section 220 instead.

### Systems, Devices and Operating Systems

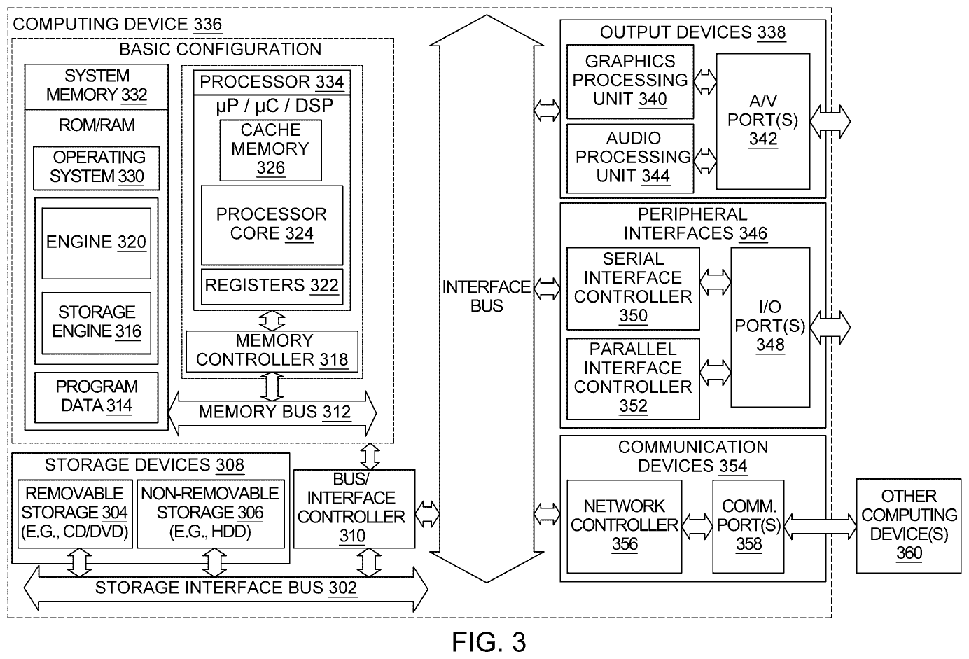

FIG. 3 illustrates a basic configuration of a computing device 336 (such as the computing device 150, 182, and in embodiments “smart device(s) 120, 110 of FIG. 1A) by those components within the inner dashed line. In the basic configuration of the computing device 336, the computing device 336 includes a processor 334 and a system memory 332. The terms “processor” and “central processing unit” or “CPU” are used interchangeably herein. In some examples, the computing device 336 may include one or more processors and the system memory 332. A memory bus 312 is used for communicating between the one or more processors 334 and the system memory 332.

Depending on the desired configuration, the processor 334 may be of any type, including, but not limited to, a microprocessor (μP), a microcontroller (μC), and a digital signal processor (DSP), or any combination thereof. In examples, the microprocessor may be AMD's ATHLON, DURON and/or OPTERON; ARM's application, embedded and secure processors; IBM and/or MOTOROLA's DRAGONBALL and POWERPC; IBM's and SONY's Cell processor; INTEL'S CELERON, CORE (2) DUO, ITANIUM, PENTIUM, XEON, and/or XSCALE; and/or the like processor(s).

Further, the processor 334 may include one or more levels of caching, such as a level cache memory 326, a processor core 324, and registers 322, among other examples. The processor core 324 may include an arithmetic logic unit (ALU), a floating point unit (FPU), and/or a digital signal processing core (DSP Core), or any combination thereof. A memory controller 318 may be used with the processor 334, or, in some implementations, the memory controller 318 may be an internal part of the memory controller 318.

Depending on the desired configuration, the system memory 332 may be of any type, including, but not limited to, volatile memory (such as RAM), and/or non-volatile memory (such as ROM, flash memory, etc.), or any combination thereof. The system memory 332 includes an operating system 330, one or more engines, such as an engine 320, and program data 314. In some embodiments, the engine 320 may be an application, a software program, a service, or a software platform, as described infra. The system memory 332 may also include a storage engine 316 that may store any information of data disclosed herein.

The operating system 330 may be a highly fault tolerant, scalable, and secure system such as: APPLE MACINTOSH OSX (Server); AT&T PLAN 9; BE OS; UNIX and UNIX-like system distributions (such as AT&T's UNIX; BERKLEY SOFTWARE DISTRIBUTION (BSD) variations such as FREEBSD, NETBSD, OPENBSD, and/or the like; Linux distributions such as RED HAT, UBUNTU, and/or the like); and/or the like operating systems. However, more limited and/or less secure operating systems also may be employed such as APPLE MACINTOSH OS, IBM OS/2, MICROSOFT DOS, MICROSOFT WINDOWS 2000/2003/3.1/95/98/CE/MILLENNIUM/NT/VISTA/XP (Server), PALM OS, and/or the like. The operating system 330 may be one specifically optimized to be run on a mobile computing device, such as IOS, ANDROID, WINDOWS Phone, TIZEN, SYMBIAN, and/or the like.

As explained supra, the GUI of the device 336 may provide a baseline and means of accessing and displaying information graphically to users. The GUI may include APPLE MACINTOSH Operating System's AQUA, IBM's OS/2, Microsoft's WINDOWS 2000/2003/3.1/95/98/CE/MILLENNIUM/NT/XP/Vista/7 (i.e., AERO), UNIX'S X-Windows (e.g., which may include additional UNIX graphic interface libraries and layers such as K DESKTOP ENVIRONMENT (KDE), MYTHTV and GNU Network Object Model Environment (GNOME)), web interface libraries (e.g., ActiveX, AJAX, (D) HTML, FLASH, JAVA, JAVASCRIPT, etc. interface libraries such as, but not limited to, DOJO, JQUERY (UI), MOOTOOLS, PROTOTYPE, SCRIPT.ACULO.US, SWFOBJECT, or YAHOO! User Interface, any of which may be used.

Additionally, a web browser component (not shown) is a stored program component that is executed by the CPU. The web browser may be a conventional hypertext viewing application such as MICROSOFT INTERNET EXPLORER, EDGE, CHROME, FIREFOX, or NETSCAPE NAVIGATOR. SECURE WEB browsing may be supplied with 128 bit (or greater) encryption by way of HTTPS, SSL, and/or the like. Web browsers allowing for the execution of program components through facilities such as ACTIVEX, AJAX, (D) HTML, FLASH, JAVA, JAVASCRIPT, web browser plug-in APIs (e.g., FIREFOX, SAFARI Plug-in, and/or the like APIs), and/or the like. Web browsers and like information access tools may be integrated into PDAs, cellular telephones, and/or other mobile devices.

A web browser may communicate to and/or with other components in a component collection, including itself, and/or facilities of the like. Most frequently, the web browser communicates with information servers, operating systems, integrated program components (e.g., plug-ins), and/or the like; e.g., it may contain, communicate, generate, obtain, and/or provide program component, system, user, and/or data communications, requests, and/or responses. Of course, in place of a web browser and an information server, a combined application may be developed to perform similar functions of both. The combined application would similarly affect the obtaining and the provision of information to users, user agents, and/or the like from the enabled nodes of the present invention.

Moreover, the computing device 336 may have additional features or functionality, and additional interfaces to facilitate communications between the basic configuration and any desired devices and interfaces. For example, a bus/interface controller is used to facilitate communications between the basic configuration and data storage devices via a storage interface bus 302. The data storage devices may be one or more removable storage devices, one or more non-removable storage devices, or a combination thereof. Examples of the one or more removable storage devices and the one or more non-removable storage devices include magnetic disk devices (such as flexible disk drives and hard-disk drives (HDD)), optical disk drives (such as compact disk (CD) drives or digital versatile disk (DVD) drives), solid state drives (SSD), and tape drives, among others. Data stores, memories, and/or repositories 184, 138, 182 and 153 are data storage devices shown in FIG. 1.

In some embodiments, an interface bus facilitates communication from various interface devices (e.g., one or more output devices 338, one or more peripheral interfaces 346, and one or more communication devices 354) to the basic configuration via the bus/interface controller 310. Some of the one or more output devices 338 include a graphics processing unit 340 and an audio processing unit 344, which are configured to communicate to various external devices, such as a display or speakers, via one or more A/V ports 342.

The one or more peripheral interfaces 346 may include a serial interface controller 350 or a parallel interface controller 352, which are configured to communicate with external devices, such as input devices (e.g., a keyboard, a mouse, a pen, a voice input device, or a touch input device, etc.) or other peripheral devices (e.g., a printer or a scanner, etc.) via one or more I/O ports 348.

Further, the one or more communication devices 354 may include a network controller 356, which is arranged to facilitate communication with one or more other computing devices 360 over a network 210 communication link via one or more communication ports 358. The one or more other computing devices 360 include servers, the database, mobile devices, and comparable devices.

The network communication link is an example of a communication media. The communication media are typically embodied by the computer-readable instructions, data structures, program modules, or other data in a modulated data signal, such as a carrier wave or other transport mechanism, and include any information delivery media. A “modulated data signal” is a signal that has one or more of its characteristics set or changed in such a manner as to encode information in the signal. By way of example, and not limitation, the communication media may include wired media (such as a wired network or direct-wired connection) and wireless media (such as acoustic, radio frequency (RF), microwave, infrared (IR), and other wireless media). The term “computer-readable media,” as used herein, includes both storage media and communication media.

It should be appreciated that the system memory 332, the one or more removable storage devices 304, and the one or more non-removable storage devices 306 are examples of the computer-readable storage media. The computer-readable storage media is a tangible device that can retain and store instructions (e.g., program code) for use by an instruction execution device (e.g., the computing device 336). Any such computer storage media is part of the computing device 336.

The computer readable storage media/medium can be a tangible device that can retain and store instructions for use by an instruction execution device. The computer readable storage media/medium may be, for example, but is not limited to, an electronic storage device, a magnetic storage device, an optical storage device, an electromagnetic storage device, and/or a semiconductor storage device, or any suitable combination of the foregoing. A non-exhaustive list of more specific examples of the computer readable storage media/medium includes the following: a portable computer diskette, a hard disk, a random access memory (RAM), a read-only memory (ROM), an erasable programmable read-only memory (EPROM or Flash memory), a static random access memory (SRAM), a portable compact disc read-only memory (CD-ROM), a digital versatile disk (DVD), a memory stick, a floppy disk, and/or a mechanically encoded device (such as punch-cards or raised structures in a groove having instructions recorded thereon), and any suitable combination of the foregoing. A computer readable storage medium, as used herein, is not to be construed as being transitory signals per se, such as radio waves or other freely propagating electromagnetic waves, electromagnetic waves propagating through a waveguide or other transmission media (e.g., light pulses passing through a fiber-optic cable), or electrical signals transmitted through a wire.

The computer-readable instructions are provided to the processor 334 of a general purpose computer, special purpose computer, or other programmable data processing apparatus (e.g., the computing device 336) to produce a machine, such that the instructions, which execute via the processor 334 of the computer or other programmable data processing apparatus, create means for implementing the functions/acts specified in the block diagram blocks. These computer-readable instructions are also stored in a computer-readable storage medium that can direct a computer, a programmable data processing apparatus, and/or other devices to function in a particular manner, such that the computer-readable storage medium having instructions stored therein comprises an article of manufacture including instructions, which implement aspects of the functions/acts specified in the block diagram blocks.

The computer-readable instructions (e.g., the program code) are also loaded onto a computer (e.g. the computing device 336), another programmable data processing apparatus, or another device to cause a series of operational steps to be performed on the computer, the other programmable apparatus, or the other device to produce a computer implemented process, such that the instructions, which execute on the computer, the other programmable apparatus, or the other device, implement the functions/acts specified in the block diagram blocks.

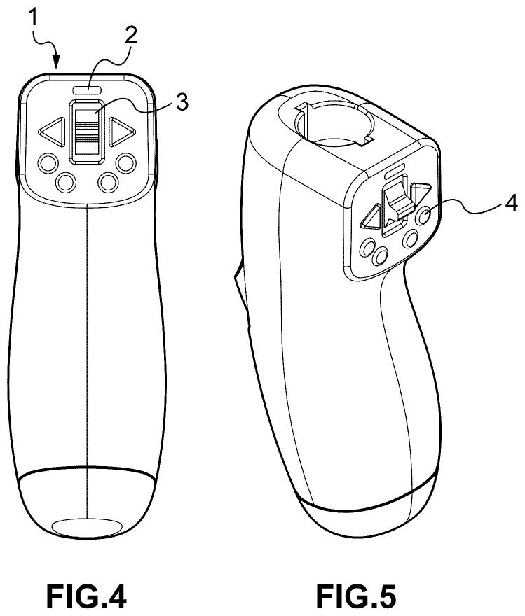

FIG. 4 illustrates another example speculum implementing the system of FIG. 1A with an unassembled housing of the smart speculum system including a housing with a handle and a control interface. The housing includes an integrated transducer and battery module 1, which is operatively configured to supply electrical power to the imaging components, heating elements, and wireless transceivers of the device. The transducer may also function as a part of a feedback or sensor circuit, capable of detecting operational status, thermal load, or patient-contact events. Disposed near the upper portion of the housing is a light indicator 2, which may include a multi-color LED or OLED display element configured to communicate system states such as active imaging, battery level, charging status, Bluetooth pairing, or error conditions. The user interface includes a width adjustment control 3, which may be embodied as a bidirectional toggle, joystick, or capacitive touch sensor, and is mechanically or electronically coupled to a blade expansion mechanism. This control allows a clinician to vary the width of an attached blade assembly with precision during an examination. In some embodiments, actuation of control 3 sends a signal to a servo or motor embedded in a removable head module, facilitating automated blade dilation.

FIG. 5 illustrates the unassembled housing of the smart speculum system of FIG. 4 including the housing with the handle, the control interface, and an opening configured to receive a support structure of a set of blades. The housing has a width adjustment control 4 and an upper interface cavity for attachment of one or more modular heads. The cavity may be shaped and keyed to receive interchangeable diagnostic modules such as an optical camera head, an ultrasound probe, or an anatomical illumination head. Electrical contacts or inductive charging coils may be located within the recessed area to provide power and data connectivity to the attached modules. The housing may further incorporate ergonomic contours to support a natural hand grip, with tactile feedback provided through the adjustment control to confirm directional input without requiring visual confirmation. The arrangement shown in FIG. 5 supports modular expandability while preserving the standalone functionality of the base unit, making it suitable for use in clinical, educational, or telehealth settings.

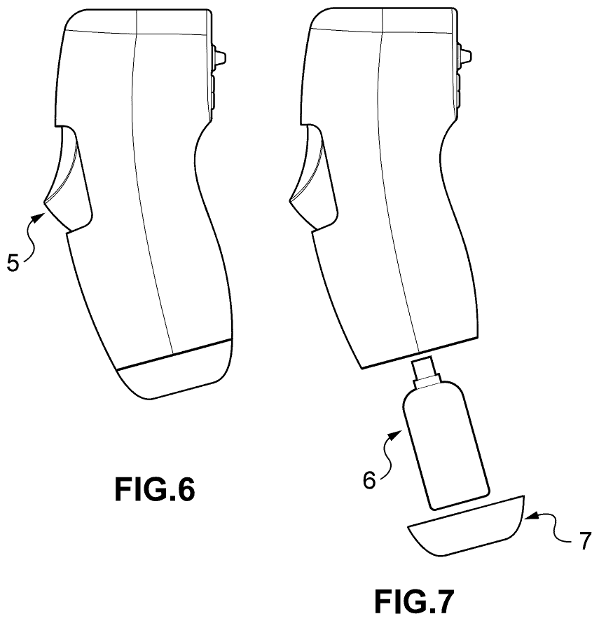

FIG. 6 illustrates the unassembled housing of the smart speculum system of FIG. 4 including the housing with the handle, the control interface, and a release mechanism with a switch to be depressed. In some embodiments a numbing spray ejector 5 integrated into the contour of the device. The numbing spray ejector is ergonomically positioned on the posterior surface of the grip portion and may be configured as a manual or electronically actuated trigger. This ejector is operatively coupled to an internal fluidic delivery channel terminating near the distal end of a removable blade head or imaging component. Upon actuation, the ejector 5 dispenses a precise dose of a topical anesthetic spray, such as lidocaine or benzocaine, directly to a target mucosal surface, such as the cervix or vaginal wall. The ejector may include dose metering, nozzle alignment features, and safety interlocks to prevent inadvertent discharge.

As used herein, the term numbing spray refers to a topical analgesic formulation configured to provide localized anesthesia by temporarily desensitizing mucosal or dermal tissue. The numbing spray is delivered in a metered aerosolized or fine mist form through an integrated ejector mechanism and is intended for application to internal anatomical surfaces such as the cervix, vaginal wall, or anal canal during diagnostic or therapeutic procedures. The formulation may include active analgesic agents such as lidocaine hydrochloride, benzocaine, tetracaine, pramoxine, or dyclonine hydrochloride. These agents act by blocking nerve signal transmission at the site of application, thereby reducing pain, discomfort, or procedural anxiety. In certain embodiments, the numbing spray is housed in a removable or replaceable refill bottle, which may be pressurized or manually actuated, and is coupled with a protective cap to prevent contamination or leakage. The formulation may further include excipients such as ethanol or water-based solvents, pharmaceutical-grade propellants, pH stabilizers, and preservatives to ensure efficacy, sterility, and mucosal compatibility.

FIG. 7 illustrates the unassembled housing of the smart speculum system of FIG. 4 including the housing with the handle, the control interface, and a release mechanism with a switch to be depressed and to eject a cap and a container retaining a topical anesthetic from a compartment of the housing, the topical anesthetic configured to be dispensed from the housing when the container is retained in the housing. A numbing spray refill bottle 6 and an associated protective lower cap 7. The refill bottle 6 is detachably inserted into the internal reservoir chamber of the housing and may utilize a click-lock or threaded interface for secure coupling. The refill bottle may be fabricated from a medical-grade polymer with pressure regulation features for consistent spray delivery. The protective cap 7 may serve as a physical shield and containment basin to prevent leakage or contamination when the refill bottle is detached or stored. In some embodiments, the refill bottle may include an RFID tag or fluid-level sensor to ensure proper identification and dosage tracking within the control software.

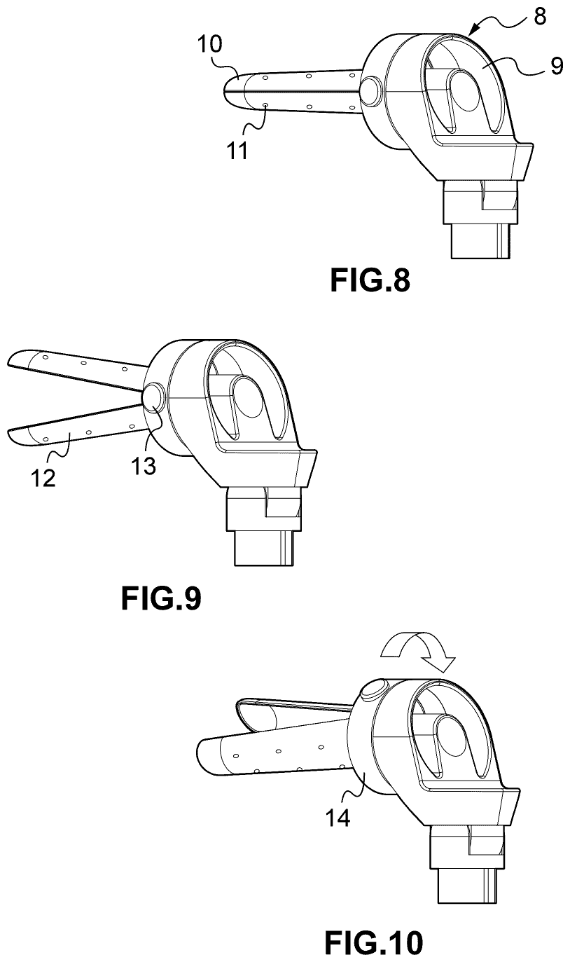

FIG. 8 is a front perspective view of a support structure connected to a set of blades oriented in a closed position, the support structure is configured to be removably connected to the housing of FIGS. 4-7. A reusable speculum head 8 is configured for detachable coupling to the handle or housing portion of the system. The speculum head may include a transparent or semi-transparent viewing window 9 that permits both visual and optical device access to internal anatomical regions, such as the cervix or vaginal canal. Integrated near the distal end of the upper blade are ultrasound sensor tips 10, which are configured to emit and receive acoustic signals for real-time sonographic imaging. These tips are positioned to remain in contact with mucosal surfaces during clinical use to ensure consistent data acquisition. The head may further comprise one or more sensors 11, such as temperature, pH, or pressure sensors, embedded along the inner blade surface. These sensors are electrically interfaced with a microcontroller embedded in the main housing and may be configured to transmit readings in real time to a connected display or processing system.

FIG. 9 is a front perspective view of the support structure of FIG. 8 connected to a set of blades oriented in an open position, the support structure has a protrusion structure configured to be retained in the opening of the housing of FIGS. 4-7. The reusable speculum head is depicted in an open configuration. The speculum head includes a set of opening blades 12, which are pivotally coupled about a hinge 13. The hinge 13 allows for smooth angular displacement of the blades to enable insertion, dilation, and visualization of the examination site. The opening mechanism may be manually or electromechanically actuated, depending on the embodiment. In electromechanical embodiments, blade actuation may be responsive to user controls located on the main housing or triggered via remote software interface.

FIG. 10 is a front perspective view of the support structure of FIG. 9 connected to a set of blades oriented in an open position, the support structure has a rotation mechanism configured to rotate the set of adjustable blade in a first direction and in a second opposite direction. The reusable speculum head is configured for rotational and/or angular adjustability. The upper portion of the head may be mounted on a rotatable collar 14 or pivot ring, allowing the speculum head to rotate about its longitudinal axis. This rotational capability enables clinicians to optimize blade orientation relative to patient anatomy and accommodate variable examination angles. In some embodiments, this rotation may be indexed or constrained by detents, locking teeth, or feedback sensors to ensure precision and repeatability. The design also facilitates improved access to various diagnostic zones and supports multi-sensor integration without compromising ergonomic control.

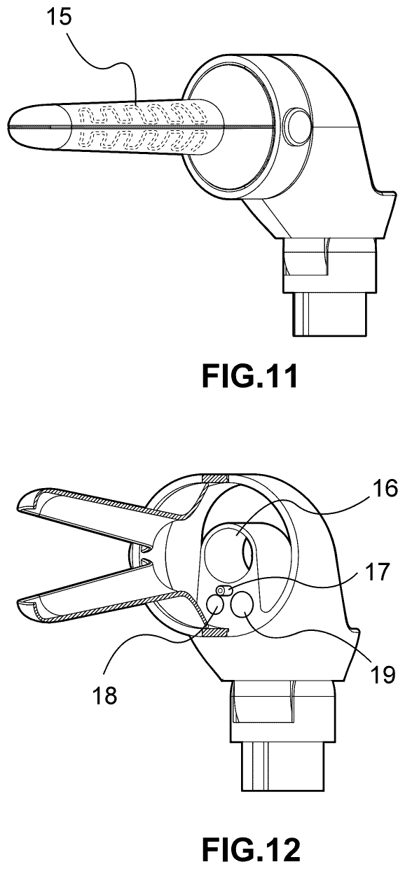

FIG. 11 is a rear perspective view of the support structure of FIG. 9 connected to a set of blades oriented in a closed position, the support structure has a pivot structure configured to orient the support structure along the y-axis and has one or more heating elements traversing at least a portion of the length of the set of blades. The reusable speculum head featuring a set of heated blades 15. The heated blades are configured to elevate their surface temperature to approximate physiological body temperature (approximately 98.6° F. or 37° C.), thereby improving patient comfort during insertion and examination. The blades may include integrated resistive heating elements embedded within medical-grade thermally conductive polymers or metal cores. In some embodiments, the heating circuitry is governed by a closed-loop temperature control system incorporating embedded sensors and a fail-safe threshold that interrupts current flow if a temperature limit is exceeded. The blades may also be coated with a hydrophobic or antimicrobial material to facilitate cleaning and minimize contamination.

FIG. 12 illustrates the support structure of FIG. 9 connected to a set of blades oriented in an open position. In some embodiments, the internal functional components support real-time imaging and comfort features. The front of the head includes an aligning guard 16 for inserting the blades smoothly and with correct angular orientation, which may be rounded or flared to facilitate atraumatic entry into the anatomical cavity. The aligning guard 16 is shown at the front, shaped as a tapered or curved leading edge designed to guide the insertion of the blades into the vaginal canal. The guard 16 may include tactile or visual alignment markers and may be composed of a low-friction biocompatible polymer to minimize resistance during insertion. In some embodiments, guard 16 may be removable or disposable, or may incorporate antimicrobial or lubricious coatings to improve hygiene and reduce tissue irritation. The geometry of the guard may also facilitate self-centering within the vaginal vault, promoting accurate blade alignment without the need for excessive manipulation.

Located just posterior to the tip is a numbing spray nozzle 17, which is coupled to an internal pressurized reservoir or cartridge (e.g., refill bottle 6 in FIG. 7). This nozzle dispenses a topical anesthetic, such as lidocaine spray, in a metered dose through a narrow jet aperture to targeted mucosal tissue, reducing discomfort during procedures such as Pap smears or biopsies. Positioned adjacent to the distal tip of the housing is a numbing spray nozzle 17, configured to deliver a fine mist or directed jet of topical anesthetic to the cervix or vaginal wall. The nozzle 17 may be made from a medical-grade elastomer or thermoplastic and is fluidly coupled to an internal pressurized canister or syringe-style refill unit (such as 6 in FIG. 7). The nozzle may incorporate a check valve or solenoid-actuated aperture to ensure precise, single-dose release in response to user activation. The spray pattern may be conical, fan-shaped, or adjustable via interchangeable nozzle tips, and in some embodiments, multiple nozzles may be positioned circumferentially for full 360-degree coverage.

A light source 18, which may comprise an LED or miniature fiber optic emitter, is positioned adjacent to the working end of the blades to illuminate the internal anatomy during inspection. A camera 19, aligned along the central optical axis of the speculum head, is embedded in the housing and configured to capture high-resolution still images or real-time video. Adjacent to the viewing window and integrated into the optical axis of the speculum head is the light source 18, such as a high-intensity white LED or a multispectral emitter capable of emitting both visible and near-infrared (NIR) wavelengths. The light source may be encased in a sealed, heat-dissipative housing to prevent overheating and may operate in conjunction with a lens or diffuser to provide even illumination across the cervix and vaginal walls. In enhanced versions, the light source may be spectrally tuned to support tissue fluorescence imaging or detection of vascularization patterns, thereby enabling adjunctive diagnostic modalities such as colposcopy or cancer screening.

In some embodiments, the camera 19 may include features such as autofocus, infrared or near-infrared imaging capabilities, and wireless transmission of the visual data to a digital twin processing system. Coaxial with the viewing axis is the camera 19, which may include a miniature CMOS or CCD image sensor with a fixed-focus or autofocus lens system. The camera is mounted in a vibration-isolated frame to preserve image stability during use and may feature digital signal processing capabilities such as real-time contrast enhancement, image stitching, and metadata tagging. The camera 19 is operatively coupled to a wireless transmitter, allowing it to stream high-definition video or still images to a base unit or cloud-based digital twin platform. In some embodiments, the camera supports synchronized capture with the light source and numbing nozzle to timestamp events and enhance correlation between anatomical visualization and intervention.

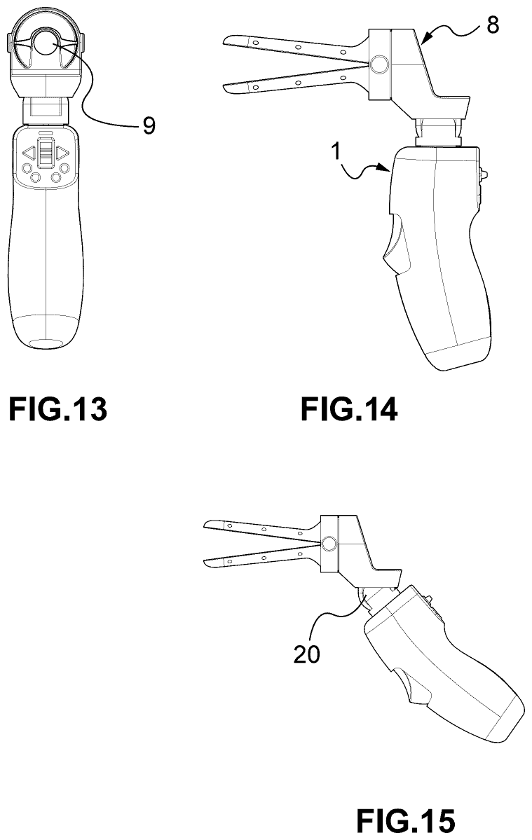

FIG. 13 illustrates the assembled housing of FIGS. 4-7 connected to the support structure of FIGS. 8-12 with the set of blades oriented in an open position, in accordance with embodiments of the present invention. A viewing window 9 is positioned at the central front portion of the reusable head, aligned with an embedded imaging system. The viewing window is composed of an optically clear medical-grade polymer or glass, and may include an anti-fog or anti-glare coating to improve visibility under clinical lighting conditions. This window facilitates both direct visual inspection and digital capture of anatomical regions such as the cervix, endocervical canal, and vaginal walls, when used in conjunction with a camera system described in FIG. 12.

FIG. 14 is a left side plan view of the housing of FIGS. 4-7 connected to the support structure of FIGS. 8-12 forming an assembled speculum with the set of blades oriented in an open position, in accordance with embodiments of the present invention. The reusable speculum head 8 may be secured to the transducer and battery module 1. The housing includes power supply and data management components used to operate heating, imaging, and communication subsystems within the head. The transducer and battery module 1 may include a rechargeable lithium-ion cell, inductive charging coil, and output terminals for supplying voltage to sensors and embedded electronics. The assembly shown in FIG. 14 supports ergonomic hand placement and provides intuitive access to user interface controls positioned on the dorsal housing surface.

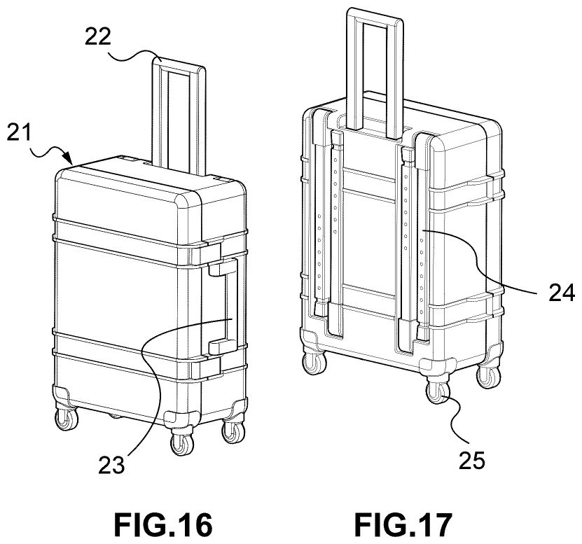

FIG. 15 illustrates the assembled speculum of FIG. 14 with a pivot structure configured to orient the set of blades and the support structure along the y-axis from about 0 degrees to about 90 degrees, in accordance with embodiments of the present invention. An angle adjustment hinge 20 is located between the reusable head and the main housing. The hinge 20 permits the angular reorientation of the speculum head relative to the handle along a vertical axis. This hinge may be spring-loaded or include a detent mechanism to lock the head at predetermined angles (e.g., 0°, 15°, 30°, etc.). In some embodiments, the hinge 20 includes a mechanical tensioner or digital encoder to provide feedback on the current orientation to a connected display system. This adjustability enables the clinician to optimize anatomical alignment for both visualization and access, particularly useful for patients with atypical cervix positioning or restricted pelvic mobility.

FIG. 16 illustrates a portable carrying case in a closed position with a telescoping handle and configured to retain the system of FIG. 1A. The portable carrying case 21 is configured to store, transport, and/or recharge the smart speculum system and its associated modular components. The portable case may be fabricated from a lightweight, durable polymer or composite shell with impact-resistant features. An adjustable handle 22, such as a telescoping suitcase-style extension arm, is provided to facilitate rolling transport over a variety of terrain. The handle is lockable in at least two positions to accommodate users of different heights. A secondary carrying handle 23 is integrated into the side wall of the case for manual lifting when rolling is not feasible, such as during stair navigation or vehicle loading. The interior of the portable case (not shown in this figure) is configured with custom-molded compartments, foam inserts, and docking ports for holding the reusable speculum heads, batteries, cables, user interface tablets, and sterilization cartridges.

FIG. 17 illustrates the portable carrying case of FIG. 16 in a closed position and having foldable legs retracted. The rear face includes a set of foldable legs 24, which are pivotally attached to the case body and may be deployed outward to stabilize the case in a vertical, upright position during use. These legs may include telescopic sections or locking hinges to support variable elevations and maintain balance on uneven surfaces. Retractable wheels 25 are positioned at the base of the case to allow both rolling movement and stationary locking. In some embodiments, the wheels 25 may include an auto-locking mechanism that activates when the foldable legs are deployed or when the system detects that the case is in operation mode. This mobile design supports the deployment of the smart speculum system in decentralized clinics, mobile health units, educational demonstrations, and remote telemedicine environments.

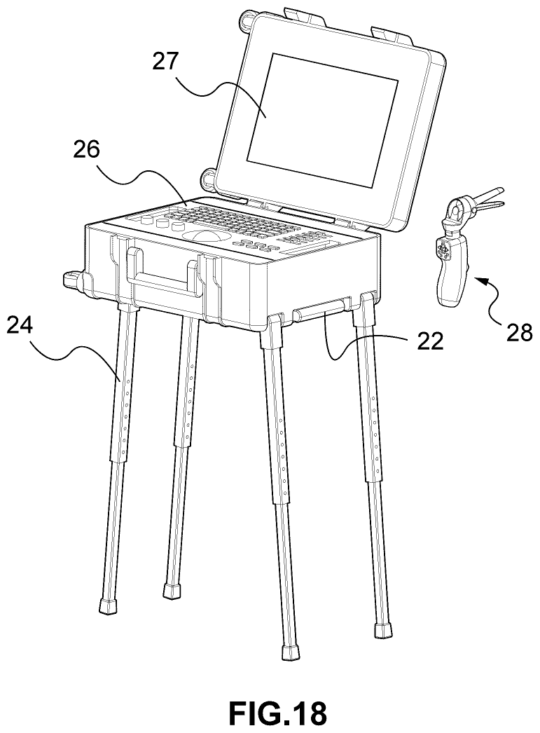

FIG. 18 illustrates the portable carrying case of FIG. 16 in an opened position with a display and a retractable control panel retained in the portable carrying case and having foldable legs extended. The case includes foldable legs 24 that are extended to support the enclosure in a free-standing configuration, allowing use in clinical, field, or mobile health environments. Extending vertically from the case is an adjustable handle 22, previously retracted for transport and now shown for identification and manipulation of the case's position. The upper surface of the case contains a retractable control panel 26, which may be stowed flush into the body of the case and deployed by mechanical or electronic release. The control panel includes user input components such as dials, buttons, trackpads, or soft membrane keys for interacting with software functions including image review, digital twin manipulation, system diagnostics, or wireless data transfer. The lid of the case includes a smart screen 27, which may be a high-resolution touchscreen display configured to present live or stored image feeds, patient data, graphical overlays, and interface dashboards. The smart screen may also support gesture or stylus input for field annotation, measurement tools, or clinician commentary.

To the right of the case is a depiction of the handheld speculum system described in earlier figures, indicated as wirelessly communicating with the case via a wireless connection 28. The wireless connection may operate over Bluetooth, Wi-Fi, or a custom radio frequency protocol to transmit imaging data, device telemetry, and control signals. In preferred embodiments, the case includes an onboard processor and memory to execute AI-based diagnostic algorithms and synchronize results to cloud services or EHR systems via encrypted connections.

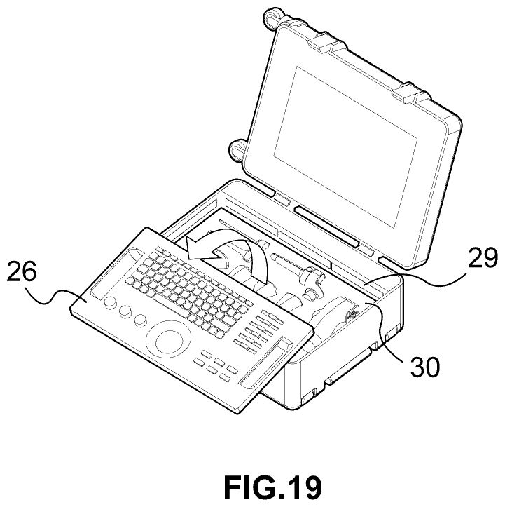

FIG. 19 illustrates the portable carrying case of FIG. 16 in an opened position with a display and a retractable control panel of FIG. 18 with a charging dock and a power source. A retractable control panel 26 slides outward from the body of the case to expose the lower storage compartment. The control panel includes integrated user interface elements and may be repositioned or collapsed to permit full access to internal subsystems housed within the base of the unit. Positioned beneath the control panel is a UV cleaning system 29, configured to perform surface sanitization of the reusable speculum head, blade tips, and other detachable components. The UV system comprises one or more ultraviolet-C (UVC) light sources oriented to illuminate the surfaces of inserted instruments, eliminating pathogens such as bacteria and viruses without the need for chemical disinfectants. The UV system may operate in an automatic cycle when the lid is closed and a sensor detects the presence of sterilizable items.

Adjacent to the UV cleaning compartment is a charging dock and battery pack 30. The dock includes contact pads or inductive coils to recharge the handheld speculum unit (shown in earlier figures) and its removable head modules. The integrated battery pack supplies power to the entire portable control station and may be hot-swappable to enable continuous operation. In preferred embodiments, the system monitors charge levels and provides real-time energy status updates to the user interface. This configuration enables the device to function independently in off-grid or low-resource environments, supporting both portability and hygiene control during clinical workflows.

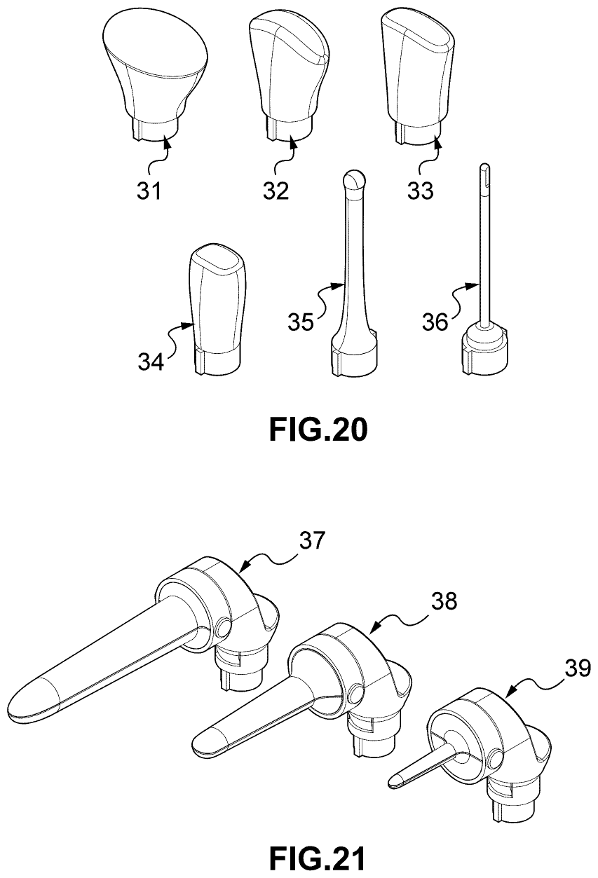

FIG. 20 illustrates a plurality of removable probe attachments configured for use with the speculum of FIG. 4. In some embodiments, a series of interchangeable ultrasound probes are compatible with the imaging and diagnostic systems described in prior figures. Each probe includes a standardized connector and housing interface for attachment to the handheld control unit or head-mounted imaging system. The depicted probes vary in geometry and internal array configuration to support different clinical imaging modalities. A 4D probe 31 enables real-time volumetric rendering by acquiring time-sequenced three-dimensional datasets, which is particularly useful for fetal development monitoring and vascular flow analysis. Adjacent to it, a convex probe 32 includes a curved transducer face that provides a wide acoustic footprint and deep tissue penetration, commonly used for abdominal or pelvic scans. A linear probe 33, featuring a flat transducer array, is suited for high-resolution superficial imaging, such as vascular studies or procedural guidance near the body surface. A phased array probe 34 is optimized for capturing rapid dynamic changes with a narrow field of view, useful in cardiology or deep-tissue penetration through confined acoustic windows. The transvaginal probe 35 is an elongated, slim-profile probe designed for intravaginal insertion and high-resolution imaging of the uterus, cervix, and adnexal structures. Finally, a rectal probe 36 is depicted, having an elongated shaft and narrow diameter, and is used for endorectal examinations including prostate evaluations or colorectal screenings. Each of these probes may incorporate RFID identification chips, sterilizable housings, or heating elements to improve comfort and workflow integration.

FIG. 21 illustrates a plurality of removable reusable speculum heads of differing sizes and configured for use with the speculum of FIG. 4. In some embodiments, at least three versions of a reusable speculum head are each tailored to different anatomical sizes and species types. A reusable speculum head 37 is shown configured for use with large mammals, including veterinary applications such as equine or bovine examinations, and features an extended length and reinforced blades for anatomical access and stability. A reusable speculum head 38 is designed for human clinical applications, including standard gynecological assessments like cervical screenings and intrauterine device placement. This version includes integrated features such as onboard lighting, heating elements, and compatibility with optical or digital imaging modules. A reusable speculum head 39 is shown configured for small mammals, including rodents or companion animals, with a minimized profile to reduce discomfort and accommodate the smaller scale of the examination area. All three heads interface with the same handheld or modular base unit and are designed for repeated sterilization using autoclave or chemical disinfection methods.

### Examples

Clause 1. A system for creating and presenting a digital twin of a patient's anatomy comprising: a speculum comprising: a set of one or more blades, which are configured to be opened or closed; at least one imaging component, wherein said imaging component is configured to generate digital images of an interior of a patient's anatomy, when the speculum is used on a patient and when the one or more blades are opened; and a transceiver configured to wirelessly transmit the digital images over a network, wherein the speculum is a portable device that lacks physical cables connecting the imaging component to an external computing device; and a digital twin system comprising a processor, memory, and transceiver, wherein said digital twin system is configured to: receive the digital images of the imaging component; to process the digital images to create a digital twin of the patient, wherein the digital twin is a visual, anatomical representation of at least a portion of the patient's anatomy examined through use of the speculum; and output the digital twin to a user of the digital twin system.
Clause 2. The system of clause 1, wherein the speculum is a two-bladed vaginal speculum, which further comprises: an upper leaf; an upper petiole; a lower leaf; a lower petiole; a hinge; and a viewing window, wherein the imaging component is positioned to not appreciably impede a physician's view via the viewing window when the speculum is used.
Clause 3. The system of clause 1, wherein the imaging component is modular and configured to be detachably coupled and interchangeably utilized with at least one speculum head or an ultrasound head, the speculum head comprising various sizes of the one or more blades and different diameters associated with an opened state of the blades.
Clause 4. The system of clause 1, wherein the at least one imaging component is an ultrasound component configured to produce ultrasound-based images.
Clause 5. The system of clause 1, wherein the speculum further comprises: a heater, which warms the set of one or more blades to within five percent of a body temperature of a patient; a fail-safe component comprising a temperature-activated limit switch configured to interrupt power to the heater when a sensed temperature exceeds a predefined safety threshold, thereby preventing the heater from overheating the set of one or more blades; and a light source configured to emit both visible light in a wavelength range of approximately 400 to 700 nanometers and near-infrared (NIR) light in a wavelength range of approximately 700 to 1000 nanometers.
Clause 6. The system of clause 1, further comprising: a case comprising a compartment configured to secure the speculum, when not in use, wherein the speculum comprises a portable power source, which is automatically charged from a power source associated with the case, when the speculum is contained in the compartment.
Clause 7. The system of clause 6, wherein the case is a portable case used to carry and secure the speculum.
Clause 8. The system of clause 6, wherein the case comprises a first and second case transceiver, wherein the transceiver of the speculum is a low-power one able to wirelessly transmit the digital images over a personal area network having a low-power range of less than 100 yards, wherein the first case transceiver receives the digital images over a connection of the personal area network, wherein the second case transceiver has a range greater than that of a low power range, which conveys the digital images to the transceiver of the digital twin system, which is further away from the speculum than the low-power range.
Clause 9. The system of clause 6, further comprising: a sanitizer, which sanitizes the speculum when placed in the compartment using one or more sanitization modalities, including but not limited to ultraviolet radiation, heat, ozone, or chemical disinfectants; and a numbing spray comprising a topical anesthetic to contact a portion of the patient's anatomy to reduce discomfort during examination or sample collection.
Clause 10. The system of clause 6, wherein the case comprises a user interface comprising: a display screen, one or more input controls, and a keyboard, the user interface being configured to display the digital images and receive user input for operating the system.
Clause 11. The system of clause 1, wherein the digital twin is a three-dimensional image.
Clause 12. The system of clause 1, wherein the digital twin is an image derived from a plurality of different ones of the digital images.
Clause 13. The system of clause 1, wherein the digital twin visually identifies at least one potential anomaly, which is automatically determined from content of the digital images.
Clause 14. The system of clause 1, wherein the digital twin system performs real time image processing of the digital images that are received in real time.
Clause 15. A system for creating and presenting a digital twin of a patient's anatomy comprising: a speculum comprising: a housing comprising: a handle; a control interface; an opening; a release mechanism configured to eject a container retaining a topical anesthetic from a compartment of the housing, the topical anesthetic configured to be dispensed from the housing when the container is retained in the housing; at least one imaging component, wherein said imaging component is configured to generate digital images of an interior of a patient's anatomy, when the speculum is used on a patient; a set of adjustable blades, the adjustable blades are configured to be oriented in an open position and a closed position; a support structure connected to the set of blades, the support structure comprising: a protrusion structure configured to be retained in the opening of the housing; and a rotation mechanism configured to rotate the set of adjustable blades; a pivot structure configured to orient the support structure along a y-axis; and a viewing window, wherein the imaging component is positioned to not appreciably impede a physician's view via the viewing window when the speculum is used; and a transceiver configured to wirelessly transmit the digital images over a low-power network, wherein the speculum is a portable device that lacks physical cables connecting the imaging component to an external computing device; a case comprising: a compartment configured to secure the speculum, when not in use, wherein the speculum comprises a portable power source, which is automatically charged from a power source associated with the case, when the speculum is contained in the compartment; a case display; a low-power transceiver configured to communicate over the low-power network to receive the digital images; a high distance transceiver configured to communicate with an internet; a digital twin system comprising a processor, memory, and transceiver, wherein said digital twin system is configured to: receive the digital images captured by the imaging component conveyed over the internet from the case to the digital twin system; to process the digital images to create a digital twin of the patient, wherein the digital twin is a visual, anatomical representation of at least a portion of the patient's anatomy examined through use of the speculum; and convey the digital twin over the internet to the case, wherein the case is further configured to represent the digital twin on the case display.
Clause 16. A system for creating and presenting a digital twin of a patient's anatomy comprising: a speculum comprising: a set of blades, which are configured to be opened or closed; at least one imaging component, wherein said imaging component is configured to generate digital images of an interior of a patient's anatomy, when the speculum is used on a patient and when the set of blades are opened; and a first low-power transceiver configured to wirelessly transmit the digital images over a personal area network, wherein the speculum is a portable device that lacks physical cables connecting the imaging component to an external computing device; a case comprising: a compartment configured to secure the speculum, when not in use, wherein the compartment is further configured to charge the speculum and sanitize the speculum using one or more sanitization modalities including one or more of ultraviolet radiation, heat, ozone, or a chemical disinfectant; a case display; and a low-power transceiver configured to communicate over the personal area network to receive the digital images.
Clause 17. The system of clause 16, wherein the personal area network is a Bluetooth network, wherein the case further comprises: an internet transceiver configured to connect the case to the internet.
Clause 18. The system of clause 16, wherein the speculum is a two-bladed vaginal speculum, which further comprises: one or more heating elements traversing at least a portion of a length of the set of blades; a temperature-activated limit switch configured to interrupt power to the one or more heating elements when a temperature measurement from a temperature sensor exceeds a predefined safety threshold, thereby preventing the heating element from overheating the set of blades; and a viewing window, wherein the imaging component is positioned to not appreciably impede a physician's view via the viewing window when the speculum is used.
Clause 19. The system of clause 16, wherein the speculum comprises a portable power source, which is automatically charged from a power source associated with the case when the speculum is contained in the compartment, wherein the case is a portable case used to carry and secure the speculum.
Clause 20. The system of clause 16, further comprising: a digital twin system comprising a processor, memory, and transceiver, wherein said digital twin system is configured to: receive the digital images of the imaging component; to process the digital images to create a digital twin of the patient, wherein the digital twin is a visual, anatomical representation of at least a portion of the patient's anatomy examined through use of the speculum; and output the digital twin to a user of the digital twin system.

Computer readable program instructions described herein can also be downloaded to respective computing/processing devices from a computer readable storage medium or to an external computer or external storage device via a network (e.g., the Internet, a local area network, a wide area network, and/or a wireless network). The network may comprise copper transmission cables, optical transmission fibers, wireless transmission, routers, firewalls, switches, gateway computers, and/or edge servers. A network adapter card or network interface in each computing/processing device receives computer readable program instructions from the network and forwards the computer readable program instructions for storage in a computer readable storage medium within the respective computing/processing device.

Computer readable program instructions for carrying out operations of the present invention may be assembler instructions, instruction-set-architecture (ISA) instructions, machine instructions, machine dependent instructions, microcode, firmware instructions, state-setting data, configuration data for integrated circuitry, or either source code or object code written in any combination of one or more programming languages, including an object oriented programming language such as Smalltalk, C++, or the like, and procedural programming languages, such as the “C” programming language or similar programming languages. The computer readable program instructions may execute entirely on the user's computer/computing device, partly on the user's computer/computing device, as a stand-alone software package, partly on the user's computer/computing device and partly on a remote computer/computing device or entirely on the remote computer or server. In the latter scenario, the remote computer may be connected to the user's computer through any type of network, including a local area network (LAN) or a wide area network (WAN), or the connection may be made to an external computer (for example, through the Internet using an Internet Service Provider). In some embodiments, electronic circuitry including, for example, programmable logic circuitry, field-programmable gate arrays (FPGA), or programmable logic arrays (PLA) may execute the computer readable program instructions by utilizing state information of the computer readable program instructions to personalize the electronic circuitry, in order to perform aspects of the present invention.

Aspects of the present invention are described herein with reference to block diagrams of methods, computer systems, and computing devices according to embodiments of the invention. It will be understood that each block and combinations of blocks in the diagrams, can be implemented by the computer readable program instructions.

The block diagrams in the Figures illustrate the architecture, functionality, and operation of possible implementations of computer systems, methods, and computing devices according to various embodiments of the present invention. In this regard, each block in the block diagrams may represent a module, a segment, or a portion of executable instructions for implementing the specified logical function(s). In some alternative implementations, the functions noted in the blocks may occur out of the order noted in the Figures. For example, two blocks shown in succession may, in fact, be executed substantially concurrently, or the blocks may sometimes be executed in the reverse order, depending upon the functionality involved. It will also be noted that each block and combinations of blocks can be implemented by special purpose hardware-based systems that perform the specified functions or acts or carry out combinations of special purpose hardware and computer instructions.

The descriptions of the various embodiments of the present invention have been presented for purposes of illustration, but are not intended to be exhaustive or limited to the embodiments disclosed. M any modifications and variations will be apparent to those of ordinary skill in the art without departing from the scope and spirit of the described embodiments. The terminology used herein was chosen to best explain the principles of the embodiments, the practical application or technical improvement over technologies found in the marketplace, or to enable others or ordinary skill in the art to understand the embodiments disclosed herein.

Although this invention has been described with a certain degree of particularity, it is to be understood that the present disclosure has been made only by way of illustration and that numerous changes in the details of construction and arrangement of parts may be resorted to without departing from the spirit and the scope of the invention.

What is claimed is:

1. A system for creating and presenting a digital twin of a patient's anatomy comprising:
a speculum comprising:
a set of one or more blades, which are configured to be opened or closed;
at least one imaging component, wherein said imaging component is configured to generate digital images of an interior of a patient's anatomy, when the speculum is used on a patient and when the one or more blades are opened; and
a transceiver configured to wirelessly transmit the digital images over a network,
wherein the speculum is a portable device that lacks physical cables connecting the imaging component to an external computing device; and
a digital twin system comprising a processor, memory, and transceiver, wherein said digital twin system is configured to:
receive the digital images of the imaging component;
to process the digital images to create a digital twin of the patient, wherein the digital twin is a visual, anatomical representation of at least a portion of the patient's anatomy examined through use of the speculum; and
output the digital twin to a user of the digital twin system.

2. A system comprising:
a medical device including one or more imaging components configured to capture anatomical data of a patient;
a processor configured to receive the anatomical data and generate a digital twin model;
an artificial intelligence engine configured to process the digital twin model to generate one or more diagnostic insights, wherein the artificial intelligence engine comprises a neural network selected from the group consisting of a convolutional neural network, a transformer model, a recurrent neural network, or a graph neural network;
wherein the artificial intelligence engine is configured to update its inference parameters based on new training data received from clinical outcomes, including via federated learning or reinforcement learning, without transmitting raw patient data to a centralized server;
wherein the diagnostic insights are presented to a user via a display interface operatively coupled to the medical device or a remote computing system;
wherein the processor is housed within a portable medical case and configured to perform low-latency inference on anatomical image data locally, and wherein cloud-based processors are further configured to perform model retraining and system-level analytics;
wherein the digital twin model is updated in real time and wherein any diagnostic medical device configured to generate, modify, or analyze a digital anatomical representation using an artificial intelligence engine falls within the scope of the claimed invention.

3. The system of claim 1, wherein the imaging component is modular and configured to be detachably coupled and interchangeably utilized with at least one speculum head or an ultrasound head, the speculum head comprising various sizes of the one or more blades and different diameters associated with an opened state of the blades.

4. The system of claim 1, wherein the at least one imaging component is an ultrasound component configured to produce ultrasound-based images.

5. The system of claim 1, wherein the speculum further comprises:
a heater, which warms the set of one or more blades to within five percent of a body temperature of a patient;
a fail-safe component comprising a temperature-activated limit switch configured to interrupt power to the heater when a sensed temperature exceeds a predefined safety threshold, thereby preventing the heater from overheating the set of one or more blades; and
a light source configured to emit both visible light in a wavelength range of approximately 400 to 700 nanometers and near-infrared (NIR) light in a wavelength range of approximately 700 to 1000 nanometers.

6. The system of claim 1, further comprising:
a case comprising a compartment configured to secure the speculum, when not in use, wherein the speculum comprises a portable power source, which is automatically charged from a power source associated with the case, when the speculum is contained in the compartment.

7. The system of claim 6, wherein the case is a portable case used to carry and secure the speculum.

8. The system of claim 6, wherein the case comprises a first and second case transceiver, wherein the transceiver of the speculum is a low-power one able to wirelessly transmit the digital images over a personal area network having a low-power range of less than 100 yards, wherein the first case transceiver receives the digital images over a connection of the personal area network, wherein the second case transceiver has a range greater than that of a low power range, which conveys the digital images to the transceiver of the digital twin system, which is further away from the speculum than the low-power range.

9. The system of claim 6, further comprising:
a sanitizer, which sanitizes the speculum when placed in the compartment using one or more sanitization modalities, including but not limited to ultraviolet radiation, heat, ozone, or chemical disinfectants; and
a numbing spray comprising a topical anesthetic to contact a portion of the patient's anatomy to reduce discomfort during examination or sample collection.

10. The system of claim 6, wherein the case comprises a user interface comprising: a display screen, one or more input controls, and a keyboard, the user interface being configured to display the digital images and receive user input for operating the system.

11. The system of claim 1, wherein the digital twin is a three-dimensional image.

12. The system of claim 1, wherein the digital twin is an image derived from a plurality of different ones of the digital images.

13. The system of claim 1, wherein the digital twin visually identifies at least one potential anomaly, which is automatically determined from content of the digital images.

14. The system of claim 1, wherein the digital twin system performs real time image processing of the digital images that are received in real time.

15. A system for creating and presenting a digital twin of a patient's anatomy comprising:
a speculum comprising:
a housing comprising:
a handle;
a control interface;
an opening;
a release mechanism configured to eject a container retaining a topical anesthetic from a compartment of the housing, the topical anesthetic configured to be dispensed from the housing when the container is retained in the housing;
at least one imaging component, wherein said imaging component is configured to generate digital images of an interior of a patient's anatomy, when the speculum is used on a patient; a set of adjustable blades, the adjustable blades are configured to be oriented in an open position and a closed position;
a support structure connected to the set of blades, the support structure comprising:
a protrusion structure configured to be retained in the opening of the housing; and
a rotation mechanism configured to rotate the set of adjustable blades;
a pivot structure configured to orient the support structure along a y-axis; and
a viewing window, wherein the imaging component is positioned to not appreciably impede a physician's view via the viewing window when the speculum is used; and
a transceiver configured to wirelessly transmit the digital images over a low-power network,
wherein the speculum is a portable device that lacks physical cables connecting the imaging component to an external computing device;
a case comprising:
a compartment configured to secure the speculum, when not in use, wherein the speculum comprises a portable power source, which is automatically charged from a power source associated with the case, when the speculum is contained in the compartment;
a case display;
a low-power transceiver configured to communicate over the low-power network to receive the digital images;
a high distance transceiver configured to communicate with an internet;
a digital twin system comprising a processor, memory, and transceiver, wherein said digital twin system is configured to:
receive the digital images captured by the imaging component conveyed over the internet from the case to the digital twin system;
to process the digital images to create a digital twin of the patient, wherein the digital twin is a visual, anatomical representation of at least a portion of the patient's anatomy examined through use of the speculum; and
convey the digital twin over the internet to the case, wherein the case is further configured to represent the digital twin on the case display.

16. A system for creating and presenting a digital twin of a patient's anatomy comprising:
a speculum comprising:
a set of blades, which are configured to be opened or closed;
at least one imaging component, wherein said imaging component is configured to generate digital images of an interior of a patient's anatomy, when the speculum is used on a patient and when the set of blades are opened; and
a first low-power transceiver configured to wirelessly transmit the digital images over a personal area network,
wherein the speculum is a portable device that lacks physical cables connecting the imaging component to an external computing device;
a case comprising:
a compartment configured to secure the speculum, when not in use, wherein the compartment is further configured to charge the speculum and sanitize the speculum using one or more sanitization modalities including one or more of ultraviolet radiation, heat, ozone, or a chemical disinfectant;
a case display; and
a low-power transceiver configured to communicate over the personal area network to receive the digital images.

17. The system of claim 16, wherein the personal area network is a Bluetooth network, wherein the case further comprises:
an internet transceiver configured to connect the case to the internet.

18. The system of claim 16, wherein the speculum is a two-bladed vaginal speculum, which further comprises:
one or more heating elements traversing at least a portion of a length of the set of blades;
a temperature-activated limit switch configured to interrupt power to the one or more heating elements when a temperature measurement from a temperature sensor exceeds a predefined safety threshold, thereby preventing the heating element from overheating the set of blades; and
a viewing window, wherein the imaging component is positioned to not appreciably impede a physician's view via the viewing window when the speculum is used.

19. The system of claim 16, wherein the speculum comprises a portable power source, which is automatically charged from a power source associated with the case when the speculum is contained in the compartment, wherein the case is a portable case used to carry and secure the speculum.

20. The system of claim 16, further comprising:
a digital twin system comprising a processor, memory, and transceiver, wherein said digital twin system is configured to:
receive the digital images of the imaging component;
to process the digital images to create a digital twin of the patient, wherein the digital twin is a visual, anatomical representation of at least a portion of the patient's anatomy examined through use of the speculum; and
output the digital twin to a user of the digital twin system.
